(19) United States
(12) Patent Application Publication
(10) Pub. No.: US 2021/0060052 A1
(43) Pub. Date: Mar. 4, 2021

(54) IN SITU METHODS OF INDUCING OF IMMUNE RESPONSE

(71) Applicant: Atossa Therapeutics, Inc., Seattle, WA (US)

(72) Inventor: Steven C. Quay, Seattle, WA (US)

(73) Assignee: Atossa Therapeutics, Inc., Seattle, WA (US)

(21) Appl. No.: 16/981,052

(22) PCT Filed: Mar. 11, 2019
(86) PCT No.: PCT/US19/21653

§ 371 Date: Sep. 15, 2020

Related U.S. Application Data
(60) Provisional application No. 62/643,618, filed on Mar. 15, 2018.

(51) Int. Cl. A61K 31/713; A61K 9/00; A61K 47/69; A61K 38/21; A61K 38/19; A61K 38/20

(52) U.S. Cl. CPC A61K 31/713; A61K 9/0041; A61K 47/6929; A61K 45/06; A61K 38/217; A61K 38/191; A61K 38/2006; A61K 38/212

(57) ABSTRACT

The present disclosure relates to intraductal methods and compositions for inducing immune response in subjects having breast cancer. The intraductally administered compositions comprise one or more bioactive agents capable of inducing in situ maturation of antigen presenting cells and migration of mature antigen presenting cells to lymph nodes. The intraductal methods and compositions induce the activation of effector immune cells, and augment tumor cell death.

### CROSS-REFERENCE TO RELATED APPLICATION

This application claims the benefit of Provisional Application No. 62/643,618, filed Mar. 15, 2018, which is expressly incorporated herein by reference in its entirety.

### BACKGROUND

Innate immune cells, such as dendritic cells (DCs), macrophages, and natural killer (NK) cells, are involved in cancer immunosurveillance by recognizing “non-self” tumor-associated antigens (TAAs). These cells prime adaptive immune cells (such as T-cells and B-cells) against the TAAs. This leads to direct and indirect anticancer effector functions, production of anti-TAA antibodies, killing of cancer cells, and subsequent immunity capable of rejecting tumor cells possessing the corresponding TAAs. Thus, priming of adaptive immune cells by innate immune cells against TAAs is a crucial milestone that is completely dependent on the antigen-presenting and antigen-sensing capabilities of the innate immune cells. The ability of antigen presenting cells (APCs) to present “non-self” TAAs properly to prime as well as to activate adaptive immune cells is an absolute pre-requisite for activation of potent anticancer immunity. APCs include dendritic cells (DCs), macrophages and B-lymphocytes (B-cells), with DCs being the “professional” APCs.

To aid in environmental sensing and carrying out rapid innate immunity-related functions, DCs possess a diverse repertoire of cell surface receptors and intracellular receptors such as various scavenging or phagocytic receptors like CD91, integrins, CD36, surface pattern recognition receptors (PRRs), toll-like receptors (TLRs), and intracellular PRRs-like NOD-like receptors (NLRs). DC-based PRRs help in detection (and subsequent DC stimulation) of danger signals like pathogen-associated molecular patterns (PAMPs) or damage-associated molecular patterns (DAMPs).

DCs are also equipped with antigen processing machinery. Classically, intracellular antigens are presented by the major histocompatibility complex (MHC) class I presentation system while extracellular antigens (captured via phagocytosis or pinocytosis) are preferentially processed for MHC class II presentation. DCs however, are capable of “cross-presenting” antigens. i.e., in DCs, the extracellular antigens can also gain access to the MHC class I presentation system while intracellular antigen fragments can also be found on the MHC class II molecules (mediated by autophagy).

In general, DCs exist in two main states, i.e., steady state immature dendritic cells (iDCs) and fully mature DCs, a classification that is partly based on changes occurring on phenotypic level and functional level. Phenotypic maturation is attained when DCs up-regulate surface co-stimulatory molecules such as CD54, CD80, CD83, CD86 and ICOS-L along with the MHC class II molecule and CD40. DCs stimulated on the functional level secrete cytokines where the balance between inflammatory or immunostimulatory cytokines (e.g., IL-12, IL-6, IL-1β) and immunosuppressive cytokines (e.g., IL-10, TGF-β) is decided by the environmental context of the presence of “self” antigens or abnormal “non-self” or “foreign” antigens such as TAA or pathogens.

In normal, healthy conditions, DCs exist in an immature or steady state (iDCs) and maintain immune tolerance by impeding adaptive immune cells from attacking host-cells that possess “self” antigens. The iDCs exhibit continuous phagocytic (endocytic) activity and continuously present “self” antigens to T-cells which are polarized to facilitate tolerance or immunosuppression instead of being polarized toward an effector state. Such iDCs typically express constitutively low levels of CD40 (Ma et al. Semin Immunol. 2009 October; 21(5): 265-272).

Such immunotolerance is actively induced and maintained through a mixture of immune checkpoint pathways and complete lack of co-stimulatory signals provided by the DCs, e.g., DC-based presentation of ligands like OX-40L, cytotoxic T-lymphocyte-associated antigen 4 (CTLA4) and programed cell death protein 1 (PD1) to T-cells causing T-cell anergy or differentiation of immunosuppressive T-cells or regulatory T-cells (Tregs).

On the other hand, when DCs encounter tumor antigens, pathogens or entities possessing PAMPs (detected in part through PRRs) they leave the function of phagocytic scavenging, degrade “non-self” entity-derived proteins in order to yield suitable antigenic peptides that are loaded on MHC class I and II molecules, upregulate costimulatory molecules, and become mature DCs assuming antigen presenting function migrating to the lymph nodes for antigen presentation to T and B cells.

An activated and mature DC thus, provides simultaneously three sets of T-cell stimulatory signals (appropriate antigen-MHC complexes (signal 1, detected by the T-cells through a complex of T-cell receptors/TCRs-CD3), phenotypic maturation ligands i.e., co-stimulatory molecules (signal 2, detected by T-cell receptors like CD28, CD40L), and suitable cytokines or factors eliciting immunostimulation and effector T-cell phenotype (signal 3, detected by respective cytokine cognate receptors), helps activate an effector profile in interacting T-cells thereby polarizing them for antigen-specific elimination of the “non-self” entity. DCs may also use other functional immunostimulatory factors in eliciting effector functions. This maturation and activation of DCs is crucial for the activation and differentiation of naïve T-cells and mounting of immune response.

In the tumor microenvironment (TME), cancer cells actively suppress steady state DCs (also called tumor-infiltrating DCs or tumor-infiltrating dendritic cell, “TIDCs”) and keep them in the tumor favoring immature state. The TIDCs state is typically characterized by: (1) the total absence or presence of negligible amounts of well-processed cancer antigens (compromised signal 1 generation), (2) absence or trivial amounts of phenotypic maturation ligands or co-stimulatory molecules (ablation of signal 2), and/or (3) either complete absence or minor presence of functional stimulus/immunostimulatory cytokines like IL-12p70 (ablated signal 3).

Among the immunoevasive strategies employed by cancer cells, one is down-regulation or loss of antigens (signal 1). Another is low expression of co-stimulatory molecule (signal 2). Lower cancer cell-associated antigen levels or low co-stimulatory molecule expression lead to unstable DC-T-cell interactions and compromised T-cell immunity. Apart from antigen and co-stimulatory molecule down-regulation, cancer cells also directly induce an immature TIDC state through secretion of immunosuppressive factors like IL-10, VEGF, TGF-β, and PGE2, thereby further compromising stable DC-T-cell interactions (signal 4).

Over the past years, DC-based vaccines have been increasingly applied in the clinical treatment of cancer patients. However, most anticancer therapies, including dendritic cell-based vaccines, tend to induce either non-immunogenic or very low-immunogenic cancer cell death disallowing sufficient DC stimulation and keeping the DCs in an immature state. For example, certain chemotherapeutics like cisplatin or certain anti-tumor vaccine-preparation methodologies like freeze thawing, may actually cause a sub-optimal activation of DCs thereby giving rise to a somewhat “limbo” state which can be termed as “semi-mature” DCs either lacking co-stimulatory signals (e.g., CD40, CD86) or suitable immunostimulatory cytokines (e.g., IL-12p70). Thus, semi-mature DCs, exhibit less than the full set of three signals required for successful/optimal T-cell activation and thereby exhibiting an unstable interface with T-cells leading to active ablation of anticancer immunity and clonal T-cell anergy. Semi-mature DCs with disparity in phenotypic maturation are able to secrete one or more of the few assorted cytokines like IL-10, IL-6, IL-10, and tumor necrosis factor (TNF) (signal 4). Together iDCs and semi-mature DCs tend to encourage T-cell anergy or T-cell exhaustion, tolerogenicity toward the cancer cell, and even active pro-tumorigenic activity.

While there have been a few attempts at in vivo maturation of DCs by systemic delivery of maturation agents like lipopolysaccharides, and cytokines such as GM-CSF (Smedt et al j. Exp. Med. 9, vol. 184, 1413-1424, 1996; Bobanga et al. Vaccines (Basel). 2013 December; 1(4): 444-462), many have attempted to generate fully mature DC's using a variety of in vitro and ex vivo methods that involve isolating a subject's blood and DC (U.S. Pat. Nos. 5,851,756, 5,994,126 and 5,475,483, 5,866,115, 6,228,640, 6,251,665, 6,121,044, 8,932,575, 7,972,847, and 8, 691,570). Studies for the treatment of breast cancer have been conducted using these dendritic cell-based vaccines prepared by in vitro or ex-vivo loading of cancer antigens onto autologous DCs (Park et al. Cancer Res Treat. 2011 March; 43(1): 56-66; Maillard et al. Cancer Research 64, 5934-5937, 2004). Such in vitro or ex-vivo preparations of DC-based vaccines face logistical and regulatory challenges related to mass production, effective preservation, shelf life, and distribution. (Kalinski et al. Immunol Res. 2011 August; 50(0): 235-247). Further, the ability to cross-present antigens differs between different DC subsets, stages of DC development and maturation, and is affected by the conditions of DC activation and maturation. While the early stages of DC maturation are in general believed to be optimal for antigen uptake and its cross-presentation, the effectiveness of tumor cell cross-presentation can be significantly affected by the selection of factors that are used in the maturation of DCs. Id.

Further, these anti-cancer therapies are often delivered intravascularly or subcutaneously. However, route of administration can affect the effectiveness of therapy (Bouvier et al. Front Immunol. 2011; 2:71). The present disclosure provides novel in situ methods for mobilizing a subject's endogenous immune cells, such as DCs, for antigen presentation and inducing immune response for fighting breast cancers obviating the need for collecting subject's blood and isolating APCs, and challenges related to ex vivo education of DCs, in vitro large production, preservation, shelf life and distribution of antigen presenting cells and dendritic cell-based vaccines. The present disclosure advantageously activates and differentiates to full maturity the tumor resident iDCs and semi-mature DCs allowing effective tumor antigens presentation to T-cells, NK cells, and B-cells, and inducing immune response and antitumor immunity.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Provided herein are methods of inducing immune response in a subject comprising administering intraductally to a breast milk duct of the subject an effective amount of a composition comprising one or more bioactive agents, wherein the composition induces in situ maturation of antigen presenting cells in the breast. In some embodiments, the one or more bioactive agents comprised in the compositions is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7- and TLR8 agonists such as imidazoquinolines (Imiquimod (3M) and Resiquimod (R848; 3M)); TLR9 agonists such as (CpG-ODNs such as PF-3512676, and the like), DAMPs such as high mobility group box-1 protein (HMGB1), cytokines (such as TNFα, IFNγ, Type I IFNs such as IFNα or IFNβ, IL-1β, IL-2, IL-12), chemokines (such as IL-1β, CCL2, or CCR7 ligands such as CCL19, CCL21), and growth factors, mi-RNA such as miR-155, costimulatory molecule agonists (such as CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropylβ-cyclodextrin, and a combination thereof.

In some embodiments, the antigen presenting cell is a dendritic cell.

In an aspect, the present disclosure provides that the compositions comprising one or more bioactive agents disclosed herein induces migration of the antigen presenting cell to a lymph node in the subject. In some embodiments, the immune response comprises activation of effector T-cells, effector NK cells, effector B-cells, or a combination thereof. In certain embodiments, the immune response comprises antitumor T-cell effector response, NK cell effector response, or B-cell anti-tumor effector response, or a combination thereof.

In some embodiments, the effector T-cells comprise cytotoxic CD8+ T-cells, CD4+ Th1 cells, memory T-cells, T follicular helper (Tfh) cells, or a combination thereof. In other embodiments, the immune response comprises reduction in immunosuppression. In at least one embodiment, the tumor size of the subject is reduced.

In an aspect, the present disclosure provides that the compositions further comprise an effective amount of a bioactive agent capable of inducing recruitment of inbound antigen presenting cells to the milk duct or breast tissue of the subject selected from the group consisting of cytokines and chemokines (such as IL-1β, MCP-1, RANTES, MIP-1α, MIP-1β, IL-8, C1q, CCL1 (CCR1 ligand), CCL2 (CCR2 ligand), CCL5 (CCR5 ligand), CCL20 (CCR6 ligand), CXCL3 (CXCR3 ligand), CXCL4 (CXCR4 ligand) and CXCL1 (CXCR1 ligand)), DAMPs such as HMGB1, and TLR agonists (like TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)), TLR4 agonists such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide, TLR7/8 agonist such as imidazoquinolines (Imiquimod (3M) and Resiquimod (R848; 3M)), TLR9 agonists such as (CpG-ODNs such as PF-3512676, and the like).

In certain embodiments, the Type-1 polarizing agent or bioactive agent capable of inducing recruitment of inbound antigen presenting cells or both, is a TLR3 agonist, TLR4 agonist, TLR7 agonist, TLR8 agonist or TLR9 agonist.

In another aspect, the present disclosure provides that the compositions further comprise an effective amount of a repolarizing agent capable of repolarizing an M2-polarized macrophage-like DC (“M2-DC”) to Type-1 polarized DC (“DC1”) selected from the group consisting of fenretinide (4-hydroxy(phenyl)retinamide, 4-HPR); IL-12; IFNγ, miR127, miR155, and miR223, ferumoxytol, inhibitors of: CSF-1, CSF-1R, IL-10, IL-10R, TGFβ, Arginase 1 (Arg1), M2 macrophage scavenger receptors (such as A, B, MARCO), histone deacetylase (HDACi), DICER, IRF4/STAT4/STAT6 signaling pathway; IL-4, IL-13, IL-17, PPARγ, KLF4, KLF6, miRNA-146 family members such as (miRNA-146a), let7 family members (such as let-7c), miRNA-9, miRNA-21, miRNA-47, miRNA-187, CCR-CC12 axis signaling, CCL2/MCP-1, placental growth factor (P1GF) (HRG) and C/EBPβ (PI3Kγ deletion), AMPKα1 (metformin), p50-p50 NFκB, NADPH oxidase (NOX) (NOX 1 and NOX 2) such as GKT137831, Rbpj, Notch signaling pathway; activators/agonists of CD40 and CD40L, IRF1, IRF5, STAT1 (such as IFNγ, vadimezan (DMXAA)) and STAT3, nuclear factor kappa B activators, toll-like receptor (TLR) agonists of TLR3, TLR4, TLR7, TLR8, and TLR9 such as Imiquimod, synthetic unmethylated cytosine-guanine (CpG) oligodeoxinucleotides (CpG-ODNs), (poly I:C), C792, lefitolimod (MGN1703), SD-101 (Dynavax), SD-101 (Dynavax), IMO-2125 (Idera); p65-p50 NFκB, MyD88, miR127, miR155, and miR223, or a combination thereof.

In another aspect, the present disclosure provides that the compositions further comprise an effective amount of a blockading agent capable of reducing or preventing antigen presenting cells from undergoing DC-to-macrophage shift, wherein the blockading agent is selected from a group consisting of CSF-1 inhibitors, CSF-1R inhibitors, MCP-1 inhibitors, IL-4 inhibitors (such as pascolizumab, pitakinra and dupilumab), IL-10 inhibitors, IL-13 inhibitors (such as anrukinzumab, lebrikizunab and tralokinumab), IL-4/IL-13 dual inhibitors such as duplimab, prostanoid inhibitors (such inhibitors of PGE3), STAT3 inhibitors (such as sorafenib, sunitinib, WP1066, and resveratrol), and STATE inhibitors (such as fenretinide (4-HPR), leflunomid, TMX264, and AS1217499), or a combination thereof.

In still another aspect, the methods comprise administering to the subject an effective amount of an additional therapeutic agent selected from the group consisting of anti-hormonals (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubicin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubincin, idarubicin, mitoxantrone, oxaliplatin, paclitaxel, bleomycin, bortezomib, oncolytic viruses, patupilone, Tyrphostin AG 490, a Janus Activated Kinase 2/signal trasducer and activator of transcription-3 (JAK2/STAT3) inhibitor, DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inibitors, anti-TGF-β inhibitors, checkpoint inhibitors (like PD-1 inhibitors such as anti-PD-1 antibodies (e.g., Nivolumab), PD-1L inhibitors such as anti-PD-1L (e.g., atezolizumab (MPDL3280), Avelumab (MSB0010718C), Durvalumab, MDX-1105), CTLA-4 inhibitors such as anti-CTLA4 antibodies (e.g., Ipilimumab), LAG-3 inhibitors such as anti-LAG-3 antibodies (e.g., IMP321, BMS-986016 and GSK2831781), OX-40 agonists such as MOXR0916, PF-04518600, MEDI0562, MEDI6469, and MEDI6383, TIM inhibitors, and IDO inhibitors), CCR4 inhibitors, FoxP3 inhibitors, cell therapy such as Chimeric Antigen Receptor/T-cell (CAR-T) therapies, and other adoptive cell therapies, or a combination thereof.

The additional therapeutic agent may be in a separate composition. In some embodiments, the additional therapeutic agent is comprised in any of the compositions comprising one or more bioactive agents, a bioactive agent capable of inducing recruitment of APCs, a repolarizing agent, or blockading agent, or any combination thereof.

In certain embodiments, the subject is intraductally administered an effective amount of a composition comprising a TLR9 agonist and an OX-40 agonist. In some embodiments, the TLR9 agonist is a CPG-ODN ranging from 0.01 μg/mL to 20 mg/mL, from 0.1 μg/mL to 15 mg/mL, from 1 μg/mL to 10 mg/mL, from 10 μg/mL to 5 mg/mL, from 50 μg/mL to 1 mg/mL per unit dose, and the OX-40 agonist antibody ranges from 0.01 mg/mL to 50 mg/mL, 0.1 mg/mL to 40 mg/mL, 0.5 mg/mL to 30 mg/mL, and 1 mg/mL to 25 mg/mL per unit dose.

In certain embodiments, the subject is intraductally administered an effective amount of a composition comprising a TLR3 agonist and IFNα. In some embodiments, the TLR3 agonist is Poly (I:C) ranging from 0.01 μg/mL to 50 μg/mL, from 0.1 μg/mL to 40 μg/mL, from 0.5 μg/mL to 25 μg/mL, and from 1 μg/mL to 20 μg/mL, and the IFNα ranges from 1 μg/mL to 300 μg/mL, from 10 μg/mL to 250 μg/mL, from 25 μg/mL to 200 μg/mL, and from 50 μg/mL to 150 μg/mL per unit dose.

In certain embodiments, the subject is administered an effective amount of a composition comprising the αDC cocktail TNFα, IL-1β, IFNγ, IFNα-2b, and Poly (I:C). In some embodiments, the TNFα ranges from 0.05 μg/mL to 150 μg/mL, from 0.1 μg/mL to 100 μg/mL, and from 0.5 μg/mL to 50 μg/mL per unit dose; IL-1β ranges from 0.01 μg/mL to 20 μg/mL, from 0.1 μg/mL to 15 μg/mL, from 0.5 μg/mL to 10 μg/mL, and from 1 μg/mL to 10 μg/mL per unit dose; IFNγ ranges from 1 μg/mL to 100 μg/mL, from 10 μg/mL to 80 μg/mL, from 25 μg/mL to 75 μg/mL, and from 50 μg/mL to 75 μg/mL; IFNα from 1 μg/mL to 300 μg/mL, from 10 μg/mL to 250 μg/mL, from 25 μg/mL to 200 μg/mL, and from 50 μg/mL to 150 μg/mL per unit dose; and Poly (I:C) ranges from 0.01 μg/mL to 50 μg/mL, from 0.1 μg/mL to 40 μg/mL, from 0.5 μg/mL to 25 μg/mL, and from 1 μg/mL to 20 μg/mL per unit dose.

In another aspect, the present disclosure provides methods of inducing migration of antigen presenting cells in a subject comprising administering intraductally to a breast milk duct of the subject an effective amount of a composition comprising one or more bioactive agents, wherein at least one bioactive agent comprised in the composition is capable of inducing migration of the antigen presenting cells to a lymph node in the subject.

In some embodiments, the one or more bioactive agents is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7 agonists and TLR8 agonists such as imidazoquinolines (Imiquimod (3M) and Resiquimod (R848; 3M)); TLR9 agonists such as (CpG-ODNs such as PF-3512676, and the like), DAMPs such as HMGB1, cytokines (such as TNFα, IFNγ, Type I IFNs such as IFNα or IFNβ, IL-1β, IL-2, IL-12p70), chemokines (such as IL-1β, MIP-3β, CCL2, CCL19, CCL21 or any CCR7 ligands), and growth factors, mi-RNA such as miR-155, costimulatory molecule agonists (such as CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropylβ-cyclodextrin, and a combination thereof.

In certain embodiments, the at least one bioactive agent capable of inducing migration of the antigen presenting cells to a lymph node in the subject is IL-1β, MIP-3β, CCL2, CCR7 ligand such as CCL19 and CCL21, LMP1, LMP1-CD40, LMP1-OX40 agonist, CD40L, MMP9, DAMPs such as HMGB1, or a combination thereof. In certain embodiments, the antigen presenting cell is a dendritic cell.

In some embodiments, the antigen presenting cells migrating to the lymph node activates cytotoxic CD8+ T-cells, CD4+ Th1 cells, memory T-cells, memory B-cells, Thf cells, NK cells, or any combination thereof. In some embodiments, the methods and compositions disclosed herein induce an anti-tumor immune response in the subject. The anti-tumor immune response comprises breast tumor infiltration by activated cytotoxic CD8+ T-cells, CD4+ Th1 cells, NK cells, B-cells or a combination thereof. In some embodiments, the size of subject's breast tumor is reduced.

In another aspect, the present disclosure provides methods for inducing or augmenting immunological cell death in breast tumor cells of a subject, comprising administering to the subject an effective amount of a cytotoxic agent, and administering intraductally an effective amount of a composition comprising one or more bioactive agents. In some embodiments, the cytotoxic agent is selected from the group consisting of temozolomide, cyclophosphamide (including low dose or metronomic cyclophosphamide), mafosfamide, doxorubicin, epirubicin, idarubicin, mitoxantrone, oxaliplatin, paclitaxel, bleomycin, bortezomib, oncolytic viruses, patupilone, tyrphostin AG 490 (JAK2/STAT3 inhibitor), or a combination thereof.

In certain embodiments, the one or more bioactive agents is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7 agonists and TLR8 agonists such as imidazoquinolines (Imiquimod (3M) and Resiquimod (R848; 3M)); TLR9 agonists such as (CpG-ODNs such as PF-3512676, and the like), cytokines (such as TNFα, IFNγ, Type I IFNs such as IFNα or IFNβ, IL-1β, IL-2, IL-12), chemokines (such as IL-1β, CCL2, CCL19, CCL21 or any CCR7 ligands), and growth factors, mi-RNA such as miR-155, costimulatory molecule agonists (such as CD-40 agonists (e.g. anti-CD40 antibodies such as RO7009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropylβ-cyclodextrin, and a combination thereof.

Under certain circumstances, it may be desirable to freshly recruit naïve APCs (“inbound APCs”) to the milk duct and the breast tissues to present tumor antigens effectively. In certain embodiments, the methods further comprise intraductal administration of an effective amount of a bioactive agent capable of inducing recruitment of inbound antigen presenting cells to the milk duct or breast tissue of the subject selected from the group consisting of cytokines and chemokines such as IL-1β, MCP-1, RANTES, MIP-1α, MIP-1β, IL-8, C1Q, CCL1 (CCR1 ligand), CCL2 (CCR2 ligand), CCL5 (CCR5 ligand), CCL20 (CCR6 ligand), CXCL3 (CXCR3 ligand), CXCL4 (CXCR4 ligand) and CXCL1 (CXCR1 ligand), DAMPs such as HMGB1, and TLR agonists (like TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)), TLR4 agonists such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide, TLR7 agonists and TLR8 agonists such as imidazoquinolines (Imiquimod (3M) and Resiquimod (R848; 3M)), TLR9 agonists such as (CpG-ODNs such as PF-3512676, (poly I:C), C792, lefitolimod (MGN1703), SD-101 (Dynavax), IMO-2125 (Idera) and the like), or a combination thereof.

In certain embodiments, the cytotoxic agent comprises oxaliplatin, and the composition comprising one or more bioactive agents comprises: (i) a TLR3 agonist poly (I:C) and IFNα; (ii) a TLR9 agonist (CpG-ODNs) and OX-40 agonist antibody; or (iii) TNFα, IFNγ, IFNα-2b, and Poly (I:C). In some embodiments, the cytotoxic agent is administered to the subject by intravenously or intraductally. The present disclosure provides that the tumor size of the subject is reduced upon intraductal administration of the cytotoxic agents and compositions comprising one or more bioactive agents.

In an aspect, the present disclosure provides that the composition is intraductally administered in a single dose or multiple doses. The composition can be administered daily, several times a day (twice, thrice, four times and the like), alternate days, every 2 days, 3 days, 5 days, 7 days, 14 days, 15 days, every 3 weeks, 28 days, monthly, quarterly, 6 monthly, and annually.

In some embodiments, the compositions further comprise an imaging agent, a dye or a contrasting agent selected from the groups consisting of gadolinium chelates, superparamagnetic iron oxide nanoparticles (SPION), ¹⁹F perfluorocarbon nanoparticles, and other magnetic reporter genes, such as metalloprotein-based MM probes.

In some embodiments, the intraductally administered compositions disclosed herein comprise a pharmaceutically acceptable carrier.

In certain embodiments, the composition is formulated as a liposome, a nanoparticle, a microparticle, a microsphere, a nanocapsule, a nanosphere, a lipid particle, a vesicle, or a micelle. In some embodiments, the one or more bioactive agents is comprised in (encapsulated) a liposome, a microparticle, a microsphere, a nanocapsule, a nanoparticle, a nanosphere, a lipid particle, a vesicle, or a micelle. In other embodiments, the one or more bioactive agents is comprised on a liposome, a microparticle, a microsphere, a nanocapsule, a nanoparticle, a nanosphere, a lipid particle, a vesicle, or a micelle. In at least one embodiment, the nanoparticle is a lipid nanoparticle.

In some embodiments, the nanoparticle is further coated with a cell targeting agent. In some embodiments, the cell targeting agent is selected from the group consisting of DEC-205, Clec9A (DNGR-1), DC-SIGN, C1q, BDCA1, BDCA2, BDCA3 and BDCA4.

In an aspect, the present disclosure provides articles of manufacture comprising a composition disclosed herein, one or more containers, packaging material, a label or package insert, and optionally, a device. In some embodiments, the device is a needle and syringe, a cannula, a catheter, a microcatheter, an osmotic pump, or an encapsulation device.

In some embodiments, the article of manufacture further comprises an additional therapeutic agent selected from the group consisting of checkpoint inhibitors, anti-hormonals (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubicin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubicin, idarubicin, mitoxantrone, oxaliplatin, paclitaxel, bleomycin, bortezomib, oncolytic viruses, patupilone, Tyrphostin AG 490 (JAK2/STAT3 inhibitor), DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inhibitors, anti-TGF-β inhibitors, checkpoint inhibitors (such as anti-PD-1 antibodies, anti-PD-1L antibodies, anti-PD-L2 antibodies, anti-CTLA-4 antibodies, anti-LAG-3 antibodies, anti-TIM-3 antibodies, and the like), anti-CCR4 inhibitors, anti-FoxP3 inhibitors, cell therapy such as Chimeric Antigen Receptor/T-cell (CAR-T) therapies, and other adoptive cell therapies, or a combination thereof.

### DETAILED DESCRIPTION

The present disclosure provides novel methods for inducing immune response in a subject having breast cancer by administering intraductally to a breast/milk duct of a subject in need thereof a composition comprising one or more bioactive agents. The bioactive agents mobilize in situ the subject's endogenous immune cells for fighting breast cancers. In contrast to the current methodologies, wherein the mobilization of immune cells in a subject requires the administration of DC-based vaccines of in vitro or ex vivo matured antigen presenting cells or ex-vivo prepared CAR-T-cells expressing chimeric antigens and costimulatory antigens, the present disclosure provides for concomitant in situ local exposure of subject's immature and/or semi-mature DC in the breast to one or more bioactive agents delivered intraductally and subject-specific tumor antigens, which results in a strong enhancement of the subsequent ability of mature APCs, such as DC to produce IL-12p70, present antigens, and to induce Th1-dominated responses (Vieira et al., J Immunol, 164: 4507-12, 2000, Maillard et al., Cancer Research, 64, 5934-5937, 2004), and more specifically the subject's cancer-specific CTL responses.

As used herein, the terms “intraductal” and “intraductally” refer to a method of treatment wherein the compositions disclosed herein are delivered to at least one breast milk duct of a subject through the ductal orifice in the nipple on the mammary papilla(e) to reach the inner depths of the breast. It will be appreciated by a person of skill in the art that intraductal methods as disclosed herein comprise delivery of the compositions disclosed herein through a natural orifice of the breast milk duct in a subject's breast. Advantageous aspects of the present disclosure are that intraductal delivery is typically effected non-invasively or minimally-invasively, does not involve deliberate breach of subject's skin or tissue or cell layer, and the compositions are delivered via the subject's own natural milk duct opening (ductal orifice) in the nipple of a mammary papilla of a subject close to the affected breast tissue.

In one aspect, intraductal administration refers to the application of the compositions of the present disclosure to the nipple of a mammary papilla, wherefrom the compositions are delivered to at least one breast milk duct through a ductal orifice in the nipple. Breast nipples and ductal orifices are uniquely suited for receiving compositions disclosed herein such as those comprising one or more bioactive agents, repolarization agents, polarizing blockading agents, cytotoxic agents, or therapeutic agents, or a combination thereof into ducts. In another aspect, intraductal administration refers to the intraductal delivery of compositions directly into the lumen of a breast milk duct via a ductal orifice in the nipple of a mammary papilla.

Breast disorders, such as breast cancers, typically originate in a milk duct of an individual (Wellings S R. Pathol. Res. Prac. 1980; 166:515-535; Love and Barsky. Cancer. 2004, vol 101(9):1947-1957). Thus, localized delivery of the therapy with compositions disclosed herein (as non-limiting example, compositions comprising one or more bioactive agents), close to the affected site within the breast milk ducts (breast ducts) is highly desirable. Intraductal administration of such compositions provide a local, effective, easy-to-administer therapy that would obviate the side effects of systemic treatment by reducing systemic exposure to the compositions disclosed herein. This would have the added benefit of reducing the possibility/risk of on-target-off-tumor effect. Local delivery to the breast duct by intraductal administration would reduce transit time of the compositions, and provide faster exposure of the APCs such as DCs, M1-macrophages and B-cells to the compositions and the neighboring tumor cells thereby improving cytotoxic activity and efficacy while also reducing the potential for drug inactivation and/or degradation due to shorter transit.

A particular advantage of the intraductal methods disclosed herein is the in situ activation and maturation of the APCs upon exposure to subject-specific tumor antigens and the intraductally delivered one or more bioactive agents concomitant with migration of the mature antigen loaded APCs to draining lymph nodes (and if present, to ectopic and tertiary lymph nodes) for antigen presentation to effector immune cells such as T-cells, NK cells and B-cells. Intraductal administration of the compositions disclosed herein induces increased antigen presentation and immune response in the subject. In various aspects, the present disclosure provides that intraductal administration of compositions disclosed herein to a breast milk duct of a subject in need thereof results in the appearance of any one or more of the following: activation and migration or mobilization of tumor specific effector immune cells such as T-cells (for example, cytotoxic CD8+ T-cells and helper T-cells (CD4+ Th1 cells)), NK cells, B-cells to a milk duct or breast tissue of the subject, increased effector functions of T-cells such as CD4+ T-helper cells, cytotoxic CD8+ T-cells, Tfh cells, and NK cells such as increased tumor cell death, increased formation of memory T-cells and B-cells, reduction in Tregs, decreased immunosuppression, and angiostatic response.

Bioactive agents suitable for inducing immune response in a subject with breast cancer include but are not limited to Type I polarizing agents forming immunostimulatory APCs that activate T-cells (“DC1”), such as TLR agonists (e.g., TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7 agonists and TLR8 agonists such as imidazoquinolines (Imiquimod (3M) and Resiquimod (R848; 3M)); TLR9 agonists such as (CpG-ODNs such as PF-3512676, and the like), DAMPs such as HMGB1, cytokines (such as TNFα, IFNγ, Type I IFNs such as IFNα or IFNβ, IL-1β, IL-2, IL-12), chemokines, and growth factors, mi-RNA, costimulatory molecule agonists (such as CD28 agonists, CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), 2-hydroxypropylβ-cyclodextrin, and the like.

In certain embodiments, the composition comprises a Type I IFN such as such as IFNα or IFNβ and a TLR3 agonist. In at least one embodiment, the composition comprises IFNα-2b and a TLR3 agonist such as Polyinosine-Polycytidine (PolyI:C). In another embodiment, the composition comprises as bioactive agents TNFα, IL-1β, and IFNγ.

In certain embodiments, the composition comprising one or more Type 1 polarizing agents further comprises IFNα and/or Poly(I:C).

In certain embodiments, the composition comprises a cocktail of Type 1 polarizing agents (the “αDC cocktail” or “α-type 1 polarizing agents”) comprising IL-1, TNF-α, IFN-α, IFN-γ, and Poly(I:C). In at least one embodiment, the αDC cocktail comprises TNFα/II-1β/IFNγ/IFNα-2b/Poly I:C. The α-Type I polarizing agents or αDC cocktail induce full maturation of DCs to form Type 1 polarized DCs (“DC1”) which: activate effector cells such as cytotoxic T-lymphocytes (CD8+ T-cells; “CTLs”) and CD4+ Th1 helper T-cells (“CD4+ Th1 cells”), are responsive to secondary lymphoid organ chemokines, and produce high interleukin-12p70 (IL-12p70)-producing ability. The αDC cocktail induces maturation of DCs to form DC that are very responsive to CD40-ligand (CD40L) signaling, based on heightened production of IL-12p70 by the mature DC1s. CD4+ T helper-1 cells (CD4+ Th1 cells) and CD8+ T-cells express CD40L and this interaction with CD40 on the DC1s appears important for amplifying and sustaining Th1-biased immunity in vivo.

In some embodiments, the compositions comprise as bioactive agents TLR9 ligands such as CpG-ODNs and CD134 (OX-40) agonists such as agonistic anti-OX-40 antibody (e.g. MOXR0916, PF-04518600, MEDI0562, MEDI6469, and MEDI6383) or inducers of OX-40L. OX-40 is a co-stimulatory molecule providing costimulatory signals that are necessary for a long-lasting immune response and for generating immune T-cell memory. OX40 signals culminate in enhanced T-cell activation, prolonged T-cell survival, generation of a memory response, prevention of T-cell tolerance, and reduction of the immunosuppressive activity of regulatory T-cells (Croft et al. Immunol Rev. 2009 May; 229(1): 173-191; Sagiv-Barfi et al. Science Translational Medicine 31 Jan. 2018:Vol. 10, Issue 426, eaan4488).

In at least one embodiment, the compositions comprise TLR9 ligand CpG-ODNs, IL-12 (such as IL-12p70), and an agonist of OX-40.

The CpG-ODNs may be of Class A, B and/or C (Krieg et al. J Clin Invest. 2007 May 1; 117(5): 1184-1194). Class A (type D) CpG ODNs (CpG-A ODNs) can contain a central palindromic unmethylated phosphodiester (PO) CpG sequence and a PS-modified 3′ poly-G tail with EC₅₀ of 1.5 μM. Class A CpG-ODNs stimulate APCs such as pDC and induce IFN-α production. Exemplary Class A CpG-ODNs include ODN 2216 (5′-ggGGGACGA:TCGTCgggggg-3′ (20 mer)). Class B CpG-ODN can contain a full unmethylated nuclease resistant phosphorothioate (PS) backbone with one or more CpG dinucleotides with EC₅₀ of 0.4 μM, such as ODN 7909 (CpG2006; 5′-tcgtcgttttgtcgttttgtcgtt-3′ (24 mer)). Class B CpG-ODNs strongly activate B cells but weakly stimulate IFN-α secretion in pDCs. Class C CpG-ODNs can contain a complete unmethylated nuclease resistant phosphorothioate (PS) backbone and a palindromic motif (CpG-ODNs), which is capable of mobilizing APCs such as pDCs as well as B-cells with EC₅₀ of 0.8 μM. Such TLR9 CpG-ODNs are capable of inducing IFNα production from pDC and B-cell stimulation and IFNα-mediated T-cell activation. Exemplary Class C CpG-ODNs include, but are not limited to, M362 (5′-tcgtcgtcgttc:gaacgacgttgat-3′ (25 mer)) or CpG 2395 (5′-tcgtcgttttcggcgc:gcgccg-3′ (22 mer)).

In some embodiments, the TLR agonist may be oligonucleotides from dSLIM family, for example, MGN 1703 (Witting et al. Critical Reviews in Oncology/Hematology. Vol. 94, Issue 1, April 2015, Pages 31-44).

In other embodiments, the intraductally delivered composition comprising one or more bioactive agents such as IFN-α and monophosphoryl lipid A (MPL).

The bioactive agents disclosed herein induce in situ the subjects' immature and semimature APCs to full maturity. Accordingly, in an aspect, the present disclosure advantageously provides that the methods and compositions disclosed herein induce in situ to full maturity subject's own APCs that are immature and/or semimature to full maturity to form immunostimulatory antigen loaded APCs (DC1s) on coming in contact with intraductally administered composition comprising one or more bioactive agents disclosed herein and tumor antigens in the breast tumor in a subject.

In some embodiments, the immune cells that are mobilized are APCs.

In some embodiments, the APCs are breast tissue resident APCs or breast tumor associated APCs, or both. For the purpose of the present disclosure, APCs can be any APC known in the art such as DCs, macrophages such as M1-polarized macrophages, and B-cells. In some embodiments, the APC is a DC or a progenitor thereof. The DCs may be any immature or steady state DC (iDC). The DCs may be bone-marrow derived, spleen-derived, or monocyte-derived DC. In some embodiments, the APC is a breast tissue-resident DC or a tumor associated DC such as a TIDC. DC subsets are known in the art and are identifiable by various markers and methods (Collins et al. Immunology. 2013 September; 140(1): 22-30; Worah et al., 2016, Cell Reports 16, 2953-2966). DCs include, but are not limited to myeloid or conventional DCs (mDCs; such as BDCA-3+ (CD141+) DCs, BDCA1+ (CD1c+) DCs), plasmacytoid DCs (pDCs; such as BDCA-2+ DCs), CD14+ DCs, langerhans cells (LC), and the like. Examples of tumor associated DC include, but are not limited to, TIDCs such as a tumor associated mDC, a tumor associated monocyte-derived DC, a tumor associated pDC, a tumor associated iDC or a tumor associated semi-mature DC. One of skill in the art will understand that DC nomenclature has been and will continue to evolve and that the present disclosure includes all DCs however identified.

Studies have shown that APCs in tumors are functionally defective and contribute to poor antitumor immune responses (Palucka et al. Cancer J. 2013; 19(6)). Tumors abuse myeloid plasticity to re-direct DC differentiation from T-cell stimulatory DC subsets (DC is) to immunosuppressive DC subsets (regulatory DCs) that can interfere with anti-tumor immunity (Gabrilovich et al. Nat Rev Immunol (2012) 12:253-68; Janco et al. J Immunol. 2015 Apr. 1; 194(7): 2985-2991). Studies have also shown that tumor cells can also directly induce an immature TIDC state through secretion of immunosuppressive factors like iNOS, IL-6, VEGF, TGF-β, and Prostaglandin-E2 (PGE2), thereby further compromising stable DC-T-cell interactions. For example, tumor-derived suppressive factors such as Arginase, PGE2, and IL-10 have been identified as factors in cultures of primary human tumors responsible for the inhibited development and activation of skin DC as well as monocyte-derived DC (Sombroek et al. J Immunol (2002) 168:4333-43).

Further, IL-10 is immunosuppressive, in that it is able to induce a “DC-to-macrophage” shift, i.e., convert fully developed DC to immature macrophage-like cells with functional M2 characteristics with an immature CD14⁺BDCA3⁺DC-SIGN⁺CD16⁺ phenotype and macrophage-like morphology, a disturbed balance in the release of immunosuppressive IL-10 (high) vs. immunostimulatory IL-12p70 (low), high expression levels of the T-cell-inhibitory molecule B7-H1/PDL-1, and lower priming efficiency of allogeneic Th cells and of CD8⁺ (cytotoxic) T-cells, binding epitope/MHC complexes with low avidity (De Gruijl et al. J Immunol (2006) 176:7232-42 Fortsch D et al. J Immunol 2000, 165:978-987; Gerlini G, et al. Am J Pathol (2004) 165:1853-63; van de Ven et al. Front. Immunol., 25 Nov. 2013). These immature and semimature TIDC thus take on M2-macrophage like polarization state (“M2-macrophage-like DCs” or “M2-DCs”) with immunosuppressive functions much like the tumor associated M2-polarized macrophages resulting in low T-cell activation (e.g. reduced cytotoxic CD8+ T-cell effector function) and reduced tumor cell death.

TIDCs, such as M2-DCs, tend to exhibit dysfunction in antigen-presenting capabilities, suppressed phagocytic and endocytic activity, abnormal motility, and various other immature characteristics. Further, tumor cells alter the TME in such a way that immunosuppressive regulatory DCs, for example CCR6 DCs and human equivalents of mice CD11b+CD11c+MHC-II+CD24+CD64lowF4/80low DCs) are recruited to tumors (Kenkel et al. Cancer Res. 2017 Aug. 1; 77(15):4158-4170).

Without wishing to be bound by any theory or mechanism, in one aspect the present disclosure provides that intraductal methods and compositions for transforming tumor associated M2-DCs and regulatory DCs to immunostimulatory DCs (DC1s).

In some embodiments, the compositions comprising one or more bioactive agents as disclosed herein comprise a repolarizing agent. Suitable repolarizing agent can include, but are not limited to, fenretinide (4-hydroxy(phenyl)retinamide, 4-HPR); IL-12; IFNγ, miR127, miR155, and miR223, ferumoxytol, inhibitors of: CSF-1, CSF-1R, IL-10, IL-10R, TGFβ, Arginase 1 (Arg1), M2 macrophage scavenger receptors (such as A, B, MARCO); histone deacetylase (HDACi), DICER, IRF4/STAT4/STAT6 signaling pathway; IL-4, IL-13, IL-17, PPARγ, KLF4, KLF6; miRNA-146 family members such as (miRNA-146a), let7 family members (such as let-7c), miRNA-9, miRNA-21, miRNA-47, miRNA-187; CCR-CC12 axis signaling; CCL2/MCP-1 synthesis; placental growth factor (P1GF) (HRG) and C/EBPβ (PI3Kγ deletion); AMPKal (metformin), p50-p50 NFκB, NADPH oxidase (NOX) (NOX 1 and NOX 2), Rbpj, Notch signaling pathway; activators of CD40 and CD40L; IRF1, IRF5, STAT1 (such as IFNγ, vadimezan (DMXAA)) and STAT3; nuclear factor kappa B activators, toll-like receptor (TLR) agonists of TLR3, TLR4 and TLR9 such as Imiquimod, synthetic unmethylated cytosine-guanine (CpG) oligodeoxinucleotides (CpG-ODNs), (poly I:C), C792, lefitolimod (MGN1703), SD-101 (Dynavax), SD-101 (Dynavax), IMO-2125 (Idera); p65-p50 NFκB, MyD88, miR127, miR155, and miR223, or a combination thereof. One of skill will understand that the one or more bioactive agents may also function as repolarizing agents.

In another aspect, the present disclosure provides that intraductal methods and compositions disclosed herein reduce or prevent DC-to-macrophage shift (“M2-shift”). In some embodiments, the compositions comprising one or more bioactive agents as disclosed herein comprise a blockading agent. Suitable blockading agents for preventing or reducing DC-to-macrophage M2-shift include, but are not limited to, CSF-1 inhibitors, CSF-1R inhibitors, MCP-1 inhibitors, IL-4 inhibitors (such as pascolizumab, pitakinra and dupilumab), IL-10 inhibitors, IL-13 inhibitors (such as anrukinzumab, lebrikizunab and tralokinumab), IL-4/IL-13 dual inhibitors such as duplimab, prostanoid inhibitors (such inhibitors of PGE3), STAT3 inhibitors (such as sorafenib, sunitinib, WP1066, and resveratrol), and STATE inhibitors (such as fenretinide (4-HPR), leflunomid, TMX264, and AS1217499), or a combination thereof.

Repolarization of M2-DCs to DC1s or reduction or prevention of M2-shift may be effected by co-delivery of the one or more bioactive agents disclosed herein intraductally together with a repolarization agent or a blockading agent or both, into a breast milk duct of the subject in need thereof. The one or more bioactive agents and the repolarization agent and/or blockading agent, can be comprised in a single composition or in separate compositions. Such separate compositions may be co-delivered to the milk duct of the subject in any order. For example, a composition comprising repolarization agent or blockading agent or both may be delivered within 1 m to 30, within 30 m to 6 hours, within 6 hours to 12 hours, and within 24 hours after the intraductal delivery of a composition comprising one or more bioactive agents disclosed herein. As another example, each of the separate compositions may be combined for delivery to the subject.

One of skill in the art will recognize that the intraductal methods and compositions disclosed herein can increase the ratio of immunostimulatory DC relative to immunosuppressive regulatory DCs or M2-DCs.

Under certain circumstances, it may be desirable to freshly recruit naïve APCs (“inbound APCs”) to the milk duct and the breast tissues to present tumor antigens effectively. Such inbound APCs can be peripheral APCs such as circulating APCs, bone-marrow derived APCs, spleen-derived APCs and the like. Accordingly, the present disclosure provides that in some embodiments, APCs are inbound APCs freshly or newly recruited to milk duct or breast tissue of a subject upon intraductal administration of compositions comprising one or more bioactive agents capable of recruiting or inducing migration of APCs to the milk duct of the subject. Such newly recruited APCs are exposed to subject-specific tumor antigens and induced to differentiate in situ in the milk duct and/or the breast tissue into fully mature antigen loaded APCs for effective antigen presentation and mounting immune response. Such intraductal methods allow optimal antigen uptake and tumor antigen cross presentation in situ close to the affected site. Further, the in situ methods disclosed herein obviate the need for collecting subject's blood and isolating APCs, selecting a non-subject-specific tumor antigen and challenges related to large production, preservation, shelf life and distribution of APC.

In some embodiments, the APCs are inbound DCs. Examples of inbound DC subsets include circulating naïve mDCs (such as BDCA-1+ (CD1c+), BDCA-3+ (CD141+), and BDCA-4+ DC), pDCs (such as BDCA-2+ DC), and progenitors thereof. Such inbound APCs become exposed to subject specific tumor antigens in situ and take up of tumor antigens by phagocytosis, pinocytosis and the like. The newly recruited inbound APCs can be prevented from undergoing M2-shift or from becoming regulatory DCs as described above.

Non-limiting examples of bioactive agents suitable for fresh recruitment of inbound APCs include IL-1β, MCP-1, RANTES, MIP-1α, MIP-1β, IL-8, or any combination thereof. In at least one embodiment, the composition comprises MIP3α, MIP1α and RANTES. In another embodiment, the composition comprises HMGB1, TLR agonists, like TLR3 agonists (such as Poly (I:C), polyadenosine-polyuridylic acid (poly AU) Ampligen (polyI:polyC (12)U; Hemispherx Biopharma) and Polyinosinic-Polycytidylic acid stabilized with poly-L-lysine and carboxymethylcellulose (Poly-ICLC, Hiltonol®)), TLR4 agonists such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide, TLR7/8 agonist such as imidazoquinolines (Imiquimod (3M) and Resiquimod (R848; 3M)), TLR9 agonists such as (CpG-ODNs such as PF-3512676, and the like), the bioactive agents for inducting chemotaxis of circulating iDC and peripheral tissue iDC to the breast milk duct and breast tissue. HMGB1 is a DAMP i.e., a “danger signal” released by dying or necrotic cells or secreted by activated macrophages which acts as chemoattractant for human monocyte-derived iDC. Bioactive agents for recruiting iDCs also include C1q, CCL1 (CCR1 ligand), CCL2 (CCR2 ligand), CCL5 (CCR5 ligand), CCL20 (CCR6 ligand), CXCL3 (CXCR3 ligand), CXCL4 (CXCR4 ligand) and CXCL1 (CXCR1 ligand). CXCL3 is capable of binding to CXCR3 on pDCs but not MoDC or blood DC which do not express CXCR3 (Villablanca et al. Histol Histopathol (2008) 23: 897-910).

It will be understood by one of skill in the art that bioactive agents that recruit inbound APCs can also be capable of inducing the APCs to full maturity. In some embodiments, the inbound APCs upon arrival to milk duct of a subject may be further exposed to compositions disclosed herein that specifically induce APCs to full maturity. The APCs induced to full maturity provide simultaneously three sets of T-cell stimulatory signals (appropriate antigen-MHC complexes (signal 1, detected by the T-cells through a complex of T-cell receptors/TCRs-CD3), phenotypic maturation ligands i.e., co-stimulatory molecules (signal 2, detected by T-cell receptors like CD28, CD40L, OX-40 (CD134, TNFRSF4)), and suitable cytokines or factors such as IL-12p70, IL-18, IL-23 eliciting immunostimulation and effector T-cell phenotype (signal 3, detected by respective cytokine cognate receptors like IL-12 receptor), helps activate an effector profile in interacting with T-cells thereby polarizing them for subject tumor antigen specific elimination of tumor cells. DCs may also use other functional immunostimulatory factors in eliciting effector functions via B-cells and NK cells.

In some embodiments, the mature DCs are CCR7+ DCs, CD80+ DCs, CD86+ DCs, CD40+ DCs, OX-40+ DCs, or any combination thereof. In other embodiments, the mature DCs exhibit the phenotypic markers CD11c⁺HLA-DR⁺, CD11c⁺MHCII^{high}, or CD11b⁺CD11c⁺CD86^{high}MHC-II^{high}.

DC obtained from subjects administered intraductally with compositions disclosed herein may release little or no immunosuppressive cytokines such as IL-10, TGF-β, IL-4, and IL-13. DC1s when tested for example in vitro by ELISA, may release inflammatory cytokines such as IL-18 and/or IL-23 and/or IL-27. In some embodiments, the activated mature DCs release IL-12p70. In other embodiments, the activated mature DC's release IFNα.

The maturation and/or repolarization to immunostimulatory APCs such as DC1s can be measured by determining (i) increase in one or more markers of maturation such as CD40, CD80, CD83, CD86, OX-40, and CCR7, or a combination thereof, and (ii) the increase in release of cytokines (signal 3) such as increase in levels of IL-6, IL-12p70, IL-18, IL-23, IL-27, TNFα, or a combination thereof from a sample obtained from the subject. The subject' sample can be any sample, for example, the sample can be blood, serum, plasma, tissue, nipple aspirate fluid, ductal lavage, lymphatic fluids, and the like. The level of APC-released cytokines IL-6, IL-8, IL-12p-70 and TNFα can be determined in tissue samples using any of the methods known in the art, for e.g., by ELISA, ELISPOT, and the like. In at least one embodiment, the cytokine measured is IL-12p70.

The tumor microenvironment can negatively affect migration of endogenous APCs such as DCs to lymph nodes (N. Seyfizadeh et al. Critical Reviews in Oncology/Hematology 107 (2016) 100-110). In an aspect, the present disclosure advantageously provides that intraductal methods and compositions disclosed herein induce the mature antigen loaded immunostimulatory APCs, such as DC1s, to migrate from breast tissue or breast cancers to draining lymph nodes (and if present, to ectopic nodes and tertiary nodes), for antigen presentation and priming of T-cells, NK cells and B-cells. Accordingly, in some embodiments, there is increased migration of DCs to secondary lymphatic organs (such as draining lymph nodes), ectopic and/or tertiary lymph nodes upon intraductal administration of compositions disclosed herein. In some embodiments, the APCs migrating from breast tissue to draining lymph nodes are any of the CCR7+ DCs, CD80+ APCs, CD86+ APCs, CD40+ APLCs, OX-40L+ APCs, or any combination thereof. Migration of the APCs can be according to lymph node chemokine gradient. Chemokines suitable for inducing such migration of activated APCs to lymph nodes can be IL-1β, MIP-3β, CCL2, CCR7 ligand such as CCL19, CCL21, CD40L, peptidoglycans, MMP9, DAMPs such as HMGB1, or a combination thereof.

In some embodiments, compositions inducing migration of activated APCs to lymph nodes comprise a cocktail of IL-1β, IL-6, MIP-3β as the bioactive agents.

In other embodiments, the compositions inducing migration of activated APCs to lymph nodes comprise TLR agonists such as CpG-ODNs and iquimod, chemokines such as CCL2, CCL19, CCL21 or other CCR7 ligand. In still other embodiments, the composition inducing migration of activated APCs to lymph nodes comprises CpG-ODNs and peptidoglycans as bioactive agents. In still other embodiments, the composition inducing migration of activated APCs to lymph nodes comprises CpG-ODNs and matrix metalloproteases such as MMP9 as bioactive agents. In some embodiments, the CpG-ODNs may be fused to other molecules such as Epstein Barr virus LMP1 protein or peptides, CD40 protein or peptides, OX-40 peptides and the like.

In some embodiments, methods and compositions disclosed herein induce migration of APCs (breast tissue and tumor resident APCs as well as newly recruited inbound APCs that are induced to full maturity by methods disclosed herein) by in situ transfection of the APCs with RNA vaccines resulting in expression of proteins comprising native Epstein-Barr virus LMP1 protein or peptide sequences. Such LMP1 proteins can be fusion proteins such as fusion proteins of LMP1 with co-stimulatory molecules that bind anti-tumor effector T-cells (for example, LMP1-CD40 fusion protein, LMP1-OX-40L fusion protein). In some embodiments, the RNA vaccines include a reporter RNA such as those coding for a fluorescent protein such as green or yellow fluorescent protein.

Migration of APCs may be monitored in vivo using Positron Emission Tomography (PET) and Single Photon Emission Computed Tomography (SPECT) in combination with Computed Tomography (CT), optical imaging technologies using fluorescence (ex, green fluorescence and yellow fluorescence proteins), bioluminescence, Magnetic Resonance Imaging (MRI), Cellular MRI and the like.

In an aspect, the present disclosure provides that the intraductal methods and composition disclosed herein induce increased antigen presentation by APCs to effector immune cells such as T-cells, B-cells and NK cells. In some embodiments, antigen presentation may be increased by fresh recruitment of naïve APCs and repolarization of breast tissue resident APCs and tumor tissue resident APCs.

In other embodiments, antigen presentation by inbound APCs, breast tissue resident APCs, and tumor associated APCs may be augmented by pre-treating a subject with a cytotoxic agent inducing tumor cell death. Increased tumor cell death may be by any cell death modality, for example, such as apoptosis (caspase dependent), necroptosis (initiated by TNF receptors following chemical suppression of caspases, i.e., caspase-independent), necrosis and immunologic cell death (“ICD”). Apoptotic, necroptotic and necrotic cell death modalities has been described by Belizario et al. in Mediators Inflamm. 2015; 2015: 128076; Vanden Berge et al. Mol Cell Oncol. 2015 October-December; 2(4): e975093).

Chemotherapeutic drugs generally kill tumor cells often by inducing apoptosis that can be accompanied by autophagy or, in a later stage, by cell necrosis. Immunogenicity in ICD is dictated by dying cells' exposure or release of several molecules able to activate the diverse components of the immune system, such as macrophages, NK-cells and DC (Cirone M, et al. Oncoimmunology. 2012 Oct. 1; 1(7):1218-1219; Bezu et al. Front Immunol. 2015; 6: 187).

Increased cell death generates higher amount of tumor associated antigens and molecules that aid in antigen presentation by the APCs. Dying tumor cells can emit a series of danger signals, the DAMPs, that dictate the recruitment and activation of specific myeloid immune effectors, hence triggering the first line of the innate response (Krysko et al. Nat Rev Cancer. 2012 December; 12(12):860-75). Such DAMPs include metabolic alterations (extrusion of ATP into the extracellular space), alterations of the cell surface (such as the exposure of calreticulin on the plasma membrane, and changes in the pericellular microenvironment (such as the nuclear and cellular exoduses of the chromatin-binding protein, HMGB1, that ultimately ignite an anticancer immune response (Bezu et al. Front Immunol. 2015; 6: 187). These DAMPs recruit antigen-presenting cells (APCs) to sites of active ICD and stimulate the uptake, processing, and presentation of dead cell-associated antigens, eventually resulting in the priming of an adaptive immune response. Such an immune response involves a complex hierarchy of immune effectors, including DCs such as monocyte-derived dendritic cells (DCs) producing interleukin-1β, γ/δ T-cells producing interleukin-17 and conventional CD8⁺ α/β T-cells producing interferon-γ (IFN-γ) Ma Y et al.; J. Exp. Med. 2011. Endogenous danger signals released from dying cells can bind Toll-like receptors (TLRs), to induce such innate immune response. For example, TLR3, TLR4 and TLR9 present on the surface of DCs recognizes its endogenous ligand HMGB1 (Tian et al. Nature Immunology volume 8, pages 487-496 (2007)), which is released from dying tumor cells, and this ignites a MyD88 (myeloid differentiation primary response gene)-dependent signaling pathway essential for the perception of ICD. The TLR4/MyD88 pathway elicited by HMGB1 inhibits the fusion between phagosomes and lysosomes, thereby facilitating tumor antigen processing and antigen presentation, which is required for the induction of restimulation or mobilization of cellular immune responses against cancer cells.

Cytotoxic agents may be delivered via any route of delivery, including oral, parenteral, intravenous, intraperitoneal, subcutaneous, intramuscular, and the like. In some embodiments, the cytotoxic agent may be administered intravenously or intraductally. Intraductal administration of a cytotoxic agent followed by intraductal administration of the compositions disclosed herein. In other embodiments, the subject may be dosed with a cytotoxic agent disclosed herein during or after treatment with a composition comprising one or more bioactive agents. The cytotoxic agent and compositions disclosed herein may be administered in one or more cycles. Exposure to such compositions disclosed herein advantageously sensitizes the tumor cells to cytotoxic agents delivered subsequently. Treatment with cytotoxic agents and the compositions disclosed herein increases antigen presentation by the APCs and immune response. Intraductally administered cytotoxic agent reduces any bystander effect of the due to lower systemic exposure of the cytotoxic agent.

Cytotoxic agent can be delivered alone or in combination with one or more other bioactive agents that induces IFN production, HMGB1 release and activates any one or more of the TLR3, TLR4, TLR7, TLR8, and TLR9 pathways on DCs. Examples of TLR9 agonists that can be used include, but are not limited to, anti-TLR-9 agonist antibodies and antibody fragments such as ScFvs, synthetic DNA and oligonucleotides containing CpG motifs (CpG ODNs) such as TTAGGG multimers, lefitolimod (MGN1703), SD-101 (Dynavax), and IMO-2125 (Idera) and the like.

Cytotoxic agents suitable for increasing tumor cell death can include, but is not limited to, an alkylating agent (such as temozolomide and cyclophosphamide), an anthracycline (such as doxorubicin, epirubincin, idarubicin, and the like), an anthracenedione such as mitoxantrone, a platinum drug (such as cisplatin, carboplatin, oxaliplatin, ormaplatin, enloplatin, and the like), a taxane (such as paclitaxel), an antimitotic drug, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, or a combination thereof.

Cytotoxic agents that are useful for inducing immunologic cell death include, but are not limited to, temozolomide, cyclophosphamide (including low dose or metronomic cyclophosphamide (for example, at a dose of 50 mg/d), mafosfamide, doxorubicin, epirubincin, idarubicin, mitoxantrone, oxaliplatin, paclitaxel, bleomycin, bortezomib, oncolytic viruses, patupilone, Tyrphostin AG 490, a Janus Activated Kinase 2/signal transducer and activator of transcription-3 (JAK2/STAT3) inhibitor, or a combination thereof.

Cell death may be measured by methods known in the art such as apoptosis assays for example by TUNNEL technique on tissue biopsy. Decrease in tumor size can be determined by mammography, CT-SCAN and MRI.

In an aspect, the present disclosure provides that the intraductal methods and compositions disclosed herein further comprise adjuvants. Suitable adjuvant can be mineral salts, emulsions, and liposomes, for example, PLGA liposomes, saponins, heat shock proteins such as HSP70, VSSP, BCG, and DETOX.

In an aspect, the present disclosure provides that the methods and composition disclosed herein induce immune response in the subject. Immune response can be any one or more of antibody and immune-cell mediated responses such as such as T-cell effector function (for example via activation of CD4+ helper-1 T-cells and cytotoxic CD8+ T-cells, T-cell mediated cytotoxicity), B-cell effector function such as tumor antigen targeting antibody formation, NK cell effector function, formation of memory T- and B-cells, reduction in Tregs, reduction of immunosuppression (for example via inhibiting checkpoint pathway molecules such as CTLA4, PD-1, PD1-L1, TIM, LAG3 or activating OX-40/OX-40L pathway), angiostatic response (inhibition of neovascularization), and induction or augmentation of tumor cell death (Park et al. Cancer Res Treat. 2011 March; 43(1): 56-66).

Thus, the methods disclosed herein would solve the problem of improper or insufficient antigen presentation and/or migration to secondary lymphoid organs such as lymph nodes and if present, to ectopic and tertiary lymph nodes, for antigen presentation to T-cells and B-cells. Inbound APCS, such as the newly recruited iDCs that are prevented from undergoing M2-shift or becoming M2-DCs, the breast tissue resident DCs, and tumor associated DCs would be able to process tumor antigens and become activated and present tumor antigens more efficiently. Thus, exposure to intraductally administered compositions disclosed herein would also sensitize the tumor cells to subsequent treatment with cytotoxic agents.

In an aspect, the present disclosure provides that compositions and methods disclosed herein mobilize effector immune cells. In some embodiments, the immune cells mobilized are effector cells such as T-cells, B-cells and NK cells. The tumor antigen specific T-cells, B-cells and NK cells are mobilized by mature antigen loaded APCs that migrate to draining lymph nodes, ectopic lymph nodes and tertiary lymph nodes for antigen presentation upon intraductal delivery of compositions disclosed herein.

In another aspect, the present disclosure provides methods and compositions that induce or augment the migration of infiltering anti-tumor T-cells to a milk duct or breast tissue of a subject upon intraductal administration of a composition comprising one or more bioactive agents capable of inducing or augmenting T-cell migration. Such T-cell migration can be along the afferent lymphatic system and/or in the blood. Without wishing to be bound by any theory or mechanism, in some embodiments, the compositions comprising such one or more bioactive agents include, but are not limited to, CCL17, CCL19, CCL20, CCL21, CCL22, CCL27, CXCL1, and CX3CL1 or any combination thereof. In at least one embodiment, in compositions comprising one or more bioactive agents, at least one bioactive agent induces migration of T-cells. Such T-cells can infiltrate breast tumors and aid tumor killing.

In another aspect, the present disclosure provides methods and compositions that induce or augment the migration of NK cells to a milk duct or breast tissue of a subject upon intraductal administration of a composition comprising one or more bioactive agents capable of inducing or augmenting NK cell migration. In some embodiments, the composition comprising such one or more of bioactive agents include, but are not limited to, IL-12, IL-15, IL-18, low-dose IL-2, or a combination thereof. In some embodiments, the compositions comprising one or more of bioactive agents further comprise miR-30c-1, miR-27a, miR-548q, miR362-5p, miR628-5p and miR152, or any combination thereof. Inclusion of such miRNA aids in NK-cell mediated cytotoxicity.

Infiltration of tumor tissue by activated anti-tumor effector immune cells is correlated with positive outcomes in breast cancer.

In an aspect, the present disclosure provides that the compositions comprising one or more bioactive agents disclosed herein are formulated for intraductal delivery in unit doses as described in Table 1.

TABLE 1
Exemplary Embodiments of Unit Doses of One or More
Bioactive Agents in Intraductal Compositions

| No. | Bioactive Agent | Exemplary Embodiments of Unit Doses |
|---|---|---|
| 1 | TNFα | 0.01 μg/mL to 200 μg/mL |
| 2 | IL-1β | 0.01 μg/mL to 20 μg/mL |
| 3 | IFNγ | 1 μg/mL to 100 μg/mL |
| 4 | IFNα | 1 μg/mL to 300 μg/mL |
| 5 | Poly (I:C) | 1 μg/mL to 50 μg/mL |
| 6 | CpG-ODNs | 0.01 μg/mL to 20 mg/mL |
| 7 | OX-40 agonist | 0.01 mg/mL to 50 mg/mL |

The unit dose of TNFα in intraductal compositions can range from 0.05 μg/mL to 150 μg/mL, from 0.1 μg/mL to 100 μg/mL, and from 0.5 μg/mL to 50 μg/mL. In some embodiments, the compositions comprise TNFα at unit dose of 0.1 μg/mL, 0.5 μg/mL, 1 μg/mL, 5 μg/mL, 10 μg/mL, 20 μg/mL, 25 μg/mL, 50 μg/mL, 100 μg/mL, 150 μg/mL and 200 μg/mL.

The unit dose of IL-10 in intraductal compositions can range from 0.01 μg/mL to 20 μg/mL, from 0.1 μg/mL to 15 μg/mL, from 0.5 μg/mL to 10 μg/mL, and from 1 μg/mL to 10 μg/mL. In some embodiments, the compositions comprise IL-1β at unit dose of 0.1 μg/mL, 0.5 μg/mL, 1 μg/mL, 5 μg/mL, 10 μg/mL, and 20 μg/mL.

The unit dose of IFNγ in intraductal compositions can range from 1 μg/mL to 100 μg/mL, from 10 μg/mL to 80 μg/mL, from 25 μg/mL to 75 μg/mL, and from 50 μg/mL to 75 μg/mL. In some embodiments, the compositions disclosed herein comprise IFNγ at unit dose of 1 μg/mL, 10 μg/mL, 15 μg/mL, 20 μg/mL, 25 μg/mL, 50 μg/mL, 75 μg/mL and 100 μg/mL.

The unit dose of IFNα in intraductal compositions can range from 1 μg/mL to 300 μg/mL, from 10 μg/mL to 250 μg/mL, from 25 μg/mL to 200 μg/mL, and from 50 μg/mL to 150 μg/mL. In some embodiments, the compositions disclosed herein comprise IFNα at unit dose of 1 μg/mL, 10 μg/mL, 15 μg/mL, 20 μg/mL, 25 μg/mL, 50 μg/mL, 75 μg/mL, 100 μg/mL, 125 μg/mL, 150 μg/mL, 200 μg/mL, 250 μg/mL and 300 μg/mL.

The unit dose of TLR3 agonists such as Poly (I:C) in intraductal compositions can range from 0.01 μg/mL to 50 μg/mL, from 0.1 μg/mL to 40 μg/mL, from 0.5 μg/mL to 25 μg/mL, and from 1 μg/mL to 20 μg/mL. In some embodiments, the compositions comprise Poly (I:C) at unit dose of 0.01 μg/mL, 0.1 μg/mL, 0.5 μg/mL, 1 μg/mL, 5 μg/mL, 10 μg/mL, 20 μg/mL, 25 μg/mL, 30 μg/mL, 35 μg/mL, 40 μg/mL, 45 μg/mL, and 50 μg/mL.

The unit dose of TLR9 agonists such as CpG-ODNs in intraductal compositions can range from 0.01 μg/mL to 20 mg/mL, from 0.1 μg/mL to 15 mg/mL, from 1 μg/mL to 10 mg/mL, from 10 μg/mL to 5 mg/mL, from 50 μg/mL to 1 mg/mL. In some embodiments, the compositions comprise CpG-ODNs at unit dose of 0.01 μg/mL, 0.1 μg/mL, 0.5 μg/mL, 1 μg/mL, 5 μg/mL, 10 μg/mL, 20 μg/mL, 25 μg/mL, 50 μg/mL, 100 μg/mL, 200 μg/mL, 300 μg/mL, 400 μg/mL, 0.5 mg/mL, 1 mg/mL, 2 mg/mL, 4 mg/mL, 6 mg/mL, 8 mg/mL, 10 mg/mL, 15 mg/mL, and 20 mg/mL.

The unit dose of an OX-40 agonist such as an anti-OX-40 antibody in intraductal compositions can range from 0.01 mg/mL to 50 mg/mL, 0.1 mg/mL to 40 mg/mL, 0.5 mg/mL to 30 mg/mL, and 1 mg/mL to 25 mg/mL. In some embodiments, the compositions comprise OX-40 agonists at unit dose of 0.01 mg/mL, 0.05 mg/mL, 0.1 mg/mL, 0.5 mg/mL, 1 mg/mL, 2 mg/mL, 4 mg/mL, 6 mg/mL, 8 mg/mL, 10 mg/mL, 15 mg/mL, 20 mg/mL, 25 mg/mL, 30 mg/mL, 35 mg/mL, 40 mg/mL, 45 mg/mL and 50 mg/mL.

In another aspect, the present disclosure provides that the compositions comprising one or more bioactive agents further comprises an additional therapeutic agent. Such additional therapeutic agent can be any agent useful for the treatment of a breast disorder.

Exemplary additional therapeutic agents include, without limitation, checkpoint inhibitors, anti-hormonals directed to reducing estrogen in subjects with breast cancer (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubincin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubincin, idarubicin, mitoxantrone, oxaliplatin, paclitaxel, bleomycin, bortezomib, oncolytic viruses, patupilone, Tyrphostin AG 490, a Janus Activated Kinase 2/signal trasducer and activator of transcription-3 (JAK2/STAT3) inhibitor, DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inibitors, anti-TGF-β inhibitors, checkpoint inhibitors (such as anti-PD-1 antibodies, anti-PD-1L antibodies, anti-PD-L2 antibodies, anti-CTLA-4 antibodies, anti-LAG-3 antibodies, anti-TIM-3 antibodies and the like), anti-CCR4 antibodies, anti-FoxP3 inhibitors, cell therapy such as Chimeric Antigen Receptor/T-cell (CAR-T) therapies, and other adoptive cell therapies, or a combination thereof.

Tumor infiltrating regulatory T-cells (T-regs; CD4+/CD25+/Foxp3+ T-cells) are associated with worst outcomes in breast cancer (Shou et al. BMC Cancer. 2016; 16(1): 687). Studies show that estrogen promotes immune tolerance by expanding the T-reg compartment (Prieto and Rosenstein. Immunology. 2006 May; 118(1):58-65; Polanczyk et al. International Immunology, Vol. 19, No. 3, pp. 337-343). The present disclosure advantageously provides intraductal methods and compositions for reducing T-regs in breast cancer subjects. Accordingly, in some embodiments, the additional therapeutic agent is one or more of anti-hormonals directed to reducing estrogen in subjects with breast cancer (e.g., anti-estrogen or anti-estrogen receptor), and are selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole.

In some embodiments, the additional therapeutic agent is one or more of checkpoint inhibitors include a checkpoint point inhibitor selected from the group consisting of anti-PD-1 (such as Nivolumab), anti-PD-1L (such as atezolizumab (MPDL3280), Avelumab (MSB0010718C), Durvalumab, MDX-1105), anti-CTLA4 (e.g., Ipilimumab), anti-LAG-3 (such as IMP321, BMS-986016 and GSK2831781), OX-40 agonist, TIM inhibitor, IDO inhibitor, or a combination thereof.

The present disclosure provides that the one or more bioactive agents, repolarizing agents, DC-to-macrophage blockading agents, and additional therapeutic agents used to practice the present disclosure can be, by way of non-limiting examples, small molecule inhibitors, small molecule activators, genes, DNA, polynucleotides, oligonucleotides (ODNs), aptamers, dendrimers, copy DNA (cDNA), naked RNA, messenger RNA (mRNA), microRNA (miRNA), antisense RNA (ASR), silencer RNA (siRNA), long non-coding RNAs (lncRNA), proteins, polypeptides, peptides, peptidomimetics, decoy sequences, DNA vaccines, RNA vaccines, self-amplifying mRNA replicon, antibodies (human, humanized, chimeric, monoclonal, polyclonal, monospecific, bispecific, and the like) and antibody fragments, saccharides, polysaccharides, gene therapy (such as CRISPR/Cas9-Mediated gene editing), and the like. It is to be understood by one of skill in the art, that when the present disclosure including the claims refers to antibodies, it is the intention of the applicant for scope of the antibody to include antibody fragments that can be used to practice the present disclosure.

Proteins, polypeptides and peptides used to practice the invention can be isolated from natural sources, be synthetic, or be recombinantly generated polypeptides. Peptides and proteins can be recombinantly expressed in vitro or in vivo. The proteins, peptides and polypeptides used to practice the invention can be made and isolated using any method known in the art. Proteins, polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

Sequences for the bioactive agents, repolarizing agents and DC-to-macrophage shift blockading agents (for example, Protein, polypeptide, gene, DNA, nucleotide sequences) can be determined as required from publicly and commercially available resources or known in the art such from NCBI Genbank and sequence viewer.

Proteins, peptides, and polypeptides may be conjugated to other moieties such as polyethylene glycol (PEG). For example, IFNα may be PEGylated or conjugated with asprigine-glycine-arginine.

The polynucleotides may be delivered in various forms. In some embodiments, a naked polynucleotide may be used, either in linear form, or inserted into a plasmid, such as an expression plasmid. In other embodiments, a live vector such as a viral or bacterial vector may be used. One or more regulatory sequences that aid in transcription of DNA into RNA and/or translation of RNA into a polypeptide may be present. In some instances, such as in the case of a polynucleotide that is a messenger RNA (mRNA) molecule, regulatory sequences relating to the transcription process (e.g. a promoter) are not required, and protein expression may be effected in the absence of a promoter. One of skill in the art can include suitable regulatory sequences as the circumstances require.

In some embodiments, the polynucleotide is present in an expression cassette, in which it is operably linked to regulatory sequences that will permit the polynucleotide to be expressed in the subject to which the composition of the invention is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

In another aspect, the present disclosure provides that compositions disclosed herein may further comprise a pharmaceutically acceptable carrier.

Carrier to be selected may be determined in part by the particular bioactive agent and for intraductal administration. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous, alcoholic, or hydroalcoholic solutions.

In some embodiments, the carrier of the composition may comprise a continuous phase of a hydrophobic substance, such as a liquid hydrophobic substance. The continuous phase may be an essentially pure hydrophobic substance or a mixture of hydrophobic substances. In addition, the carrier may be an emulsion of water in a hydrophobic substance or an emulsion of water in a mixture of hydrophobic substances, provided the hydrophobic substance constitutes the continuous phase.

Hydrophobic substances that are useful in the compositions as described herein are those that are pharmaceutically acceptable. The carrier is preferably a liquid but certain hydrophobic substances that are not liquids at atmospheric temperature may be liquefied, for example by warming, and are also useful in this invention. In one embodiment, the hydrophobic carrier may be a Phosphate Buffered Saline.

Oil or water-in-oil emulsions are particularly suitable carriers for use in the present disclosure. Oils should be pharmaceutically acceptable. Suitable oils include, for example, mineral oils (especially light or low viscosity mineral oil such as Drakeol® 6VR), vegetable oils (e.g., soybean oil), nut oils (e.g., peanut oil), or mixtures thereof. In an embodiment, the oil is a mannide oleate in mineral oil solution, commercially available as Montanide® ISA 51. Animal fats and artificial hydrophobic polymeric materials, particularly those that are liquid at atmospheric temperature or that can be liquefied relatively easily, may also be used.

In embodiments herein where the composition is described as being a water-free liposome suspension (“water-free”), it is possible that the hydrophobic carrier of these “water-free” compositions may still contain small quantities of water, provided that the water is present in the non-continuous phase of the carrier. For example, individual components of the composition may have bound water that may not be completely removed by processes such as lyophilization or evaporation and certain hydrophobic carriers may contain small amounts of water dissolved therein. Generally, compositions of the invention that are described as “water-free” contain, for example, less than about 10%, 9%, 8%, 7%, 6%, 5%, 4%, 3%, 2%, 1%, 0.5%, 0.1%, 0.05% or 0.01% water on a weight/weight basis of the total weight of the carrier component of the composition.

In another aspect, the present disclosure provides that composition disclosed herein are formulated as liposomes, microparticles, microspheres, nanocapsules, nanoparticles, nanospheres, lipid particles, vesicles, and micelles. Accordingly, in some embodiments, the one or more bioactive agents, repolarizing agents, blockading agents, additional therapeutic agents, or any combination thereof are comprised in (encapsulated) liposomes, microparticles, microspheres, nanocapsules, nanoparticles, nanospheres, lipid particles, vesicles, and micelles. In other embodiments, the one or more bioactive agents, repolarizing agents, blockading agents, additional therapeutic agents, or any combination thereof, are comprised on the surface of liposomes, microparticles, microspheres, nanocapsules, nanoparticles, nanospheres, lipid particles, vesicles, and micelles. In still other embodiments, the one or more bioactive agents, repolarizing agents, blockading agents, additional therapeutic agents, or any combination thereof, may comprised in and on the surface of liposomes, microparticles, microspheres, nanocapsules, nanoparticles, nanospheres, lipid particles, vesicles, and micelles.

Methods for making liposomes, nanoparticles, microparticles, microspheres, nanocapsules, nanospheres, lipid particles, vesicles, and micelles are well known in the art.

Liposomes are completely closed lipid membranes containing an entrapped aqueous volume. Liposomes may be unilamellar vesicles (possessing a single bilayer membrane) or multilamellar vesicles characterized by multimembrane bilayers, each bilayer may or may not be separated from the next by an aqueous layer. As used herein and in the claims, the term “liposomes” is intended to encompass all such vesicular structures as described above, including, without limitation, those described in the art as “niosomes”, “transfersomes” and “virosomes.” The amphiphilic structure of liposome particles enables encapsulation of both hydrophilic and hydrophobic pharmaceutical drugs.

A general discussion of liposomes can be found in Akbarzadeh et al. Nanoscale Research Letters 2013, 8:102; Gregoriadis G. Immunol. Today, 11:89-97, 1990; and Frezard, F., Braz. J. Med. Bio. Res., 32:181-189, 1999. Any suitable method for making liposomes may be used in the practice of the invention, or liposomes may be obtained from a commercial source. Liposomes are typically prepared by hydrating the liposome components that will form the lipid bilayer (e.g. phospholipids and cholesterol) with an aqueous solution, which may be pure water or a solution of one or more components dissolved in water, e.g. phosphate-buffered saline (PBS), phosphate-free saline, or any other physiologically compatible aqueous solution.

Liposome compositions may be obtained, for example, by using natural lipids, synthetic lipids, sphingolipids, ether lipids, sterols, cardiolipin, cationic lipids and lipids modified with poly (ethylene glycol) and other polymers. Synthetic lipids may include the following fatty acid constituents; lauroyl, myristoyl, palmitoyl, stearoyl, arachidoyl, oleoyl, linoleoyl, erucoyl, or combinations of these fatty acids. Lipids particularly suitable for the making liposomes include, but are not limited to, phospholipids, dioleoylphosphatidylcholine (DOPC), dioleoyl phosphatidylethanolamine (DOPE), triolein, dipalmytoilphospatidylglycerol (DPPG), hydrogenated soy phospatidylcholine (HSPC), di stearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin, cephalin, sphingomyelin, egg phosphatidylcholine (EPC), disodium succinate hexahydrate (DSH), (1,2-bis(oleoyloxy)-3-(trimethylammonio) propane) (DOTAP), 1-[2-(oleoyloxy)ethyl]-2-oleyl-3-(2-hydroxyethyl)imidazolinium chloride (DOTIM), dimyristoylphosphatidylcholine (DMPC), and dimyristoylphosphatidylglycerol (DMPG).

Cationic synthetic lipids such as DOTAP and DOTIM are useful for the preparation of cationic liposomes having an affinity for tumor vasculature and breast ducts. In some embodiments, the making liposomes, nanoparticles, microparticles, microspheres, nanocapsules, nanospheres, lipid particles, vesicles, micelles are prepared using cationic synthetic lipids.

Although any liposomes may be used in this invention, including liposomes made from archaebacterial lipids, in at least one embodiment, phospholipids and unesterified cholesterol are used in the liposome formulation. The cholesterol is used to stabilize the liposomes and any other compound that stabilizes liposomes may replace the cholesterol. Other liposome stabilizing compounds are known to those skilled in the art. For example, saturated phospholipids produce liposomes with higher transition temperatures indicating increased stability.

Phospholipids that are preferably used in the preparation of liposomes are those with at least one head group selected from the group consisting of phosphoglycerol, phosphoethanolamine, phosphoserine, phosphocholine and phosphoinositol. In some embodiments, the liposomes comprise lipids which are 94-100% phosphatidylcholine. Such lipids are available commercially in the lecithin Phospholipon® 90 G. When unesterified cholesterol is also used in liposome formulation, the cholesterol is used in an amount equivalent to about 10% of the weight of phospholipid. If a compound other than cholesterol is used to stabilize the liposomes, one skilled in the art can readily determine the amount needed in the composition.

In other embodiments, a liposome component or mixture of liposome components, such as a phospholipid (e.g. Phospholipon® 90G), dioleoylphosphatidylcholine (DOPC), triolein, dipalmytoilphospatidylglycerol (DPPG), hydrogenated soy phospatidylcholine (HSPC), di stearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin, cephalin, sphingomyelin, egg phosphatidylcholine (EPC), disodium succinate hexahydrate (DSH), dimyristoylphosphatidylcholine (DMPC), dimyristoylphosphatidylglycerol (DMPG), may be solubilized in an organic solvent, such as a mixture of chloroform and methanol, followed by filtering (e.g. a PTFE 0.2 μm filter) and drying, e.g. by rotary evaporation, to remove the solvents.

Hydration of the resulting lipid mixture may be effected by e.g., injecting the lipid mixture into an aqueous solution or sonicating the lipid mixture and an aqueous solution. During formation of liposomes, the liposome components form single bilaye.rs (unilamellar) or multiple bilayers (multilamellar) surrounding a volume of the aqueous solution with which the liposome components are hydrated.

In other embodiments, the liposomes may be combined with the carrier comprising a continuous hydrophobic phase.

If the carrier is composed solely of a hydrophobic substance or a mixture of hydrophobic substances (e.g. use of a 100% mineral oil carrier), the liposomes may simply be mixed with the hydrophobic substance, or if there are multiple hydrophobic substances, mixed with any one or a combination of them. If instead the carrier comprising a continuous phase of a hydrophobic substance contains a discontinuous aqueous phase, the carrier will typically take the form of an emulsion of the aqueous phase in the hydrophobic phase, such as a water-in-oil emulsion. Such compositions may contain an emulsifier to stabilize the emulsion and to promote an even distribution of the liposomes. Emulsifiers may be useful even if a water-free carrier is used, for the purpose of promoting an even distribution of the liposomes in the carrier. Typical emulsifiers include mannide oleate (Arlacel™ A), lecithin (e.g., S100 lecithin), a phospholipid, Tween™ 80, and Spans™ 20, 80, 83 and 85. In some embodiments, the volume ratio (v/v) of hydrophobic substance to emulsifier is in the range of about 5:1 to about 15:1. In at least one embodiment, the ratio is about 10:1.

The liposomes may be added to the finished emulsion, or they may be present in either the aqueous phase or the hydrophobic phase prior to emulsification. In some embodiments, the liposomes may be then dehydrated, such as by freeze-drying or lyophilization.

The one or more bioactive agents may be introduced at various different stages of the formulation process. More than one type of bioactive agent (and/or repolarizing agent or blockading agent) may be incorporated into the composition (for example, an antibody and a small molecule inhibitor or a siRNA and an mRNA, a polypeptide and a RNA vaccine, etc.).

In some embodiments, the one or more bioactive agents (and/or repolarizing agent or blockading agent) are present in the aqueous solution used to hydrate the components that are used to form the lipid bilayers of the liposomes (e.g. phospholipid(s) and cholesterol). In this case, the one or more bioactive agents (and/or repolarizing agent or blockading agent) will be encapsulated in the liposome, present in its aqueous interior. If the resulting liposomes are not washed or dried, such that there is residual aqueous solution present that is ultimately mixed with the carrier comprising a continuous phase of a hydrophobic substance, it is possible that additional polarizing agent or the blockading agent may be present outside the liposomes in the final product.

In a related technique, the one or more bioactive agents (and/or repolarizing agent or blockading agent) may be mixed with the components used to form the lipid bilayers of the liposomes, prior to hydration with the aqueous solution. The one or more bioactive agents may also be added to pre-formed liposomes, in which case the one or more bioactive agents may be actively loaded into the liposomes (i.e. encapsulated), or on (e.g., bound to) the surface of the liposomes or the antigen may remain external to the liposomes. In such embodiments, prior to the addition of one or more bioactive agents, the pre-formed liposomes may be empty liposomes (e.g. not containing encapsulated one or more bioactive agents and/or repolarizing agent or blockading agent) or the pre-formed liposomes may contain one or more bioactive agents incorporated into or associated with the liposomes. These steps may occur before mixing with the carrier comprising a continuous phase of a hydrophobic substance.

In an alternative approach, the one or more bioactive agents (and/or repolarizing agent or blockading agent) may instead be mixed with the carrier comprising a continuous phase of a hydrophobic substance, before, during, or after the carrier is combined with the liposomes. If the carrier is an emulsion, the one or more bioactive agents (and/or repolarizing agent or blockading agent) may be mixed with either or both of the aqueous phase or hydrophobic phase prior to emulsification. Alternatively, the one or more bioactive agents (and/or repolarizing agent or blockading agent) may be mixed with the carrier after emulsification. In some embodiments, the one or more bioactive agents (and/or repolarizing agent or blockading agent) may be present within the liposomes and also in the carrier comprising a continuous phase of a hydrophobic substance.

If the compositions comprising one or more bioactive agents further comprise additional therapeutic agents, then the additional therapeutic agents may be combined as described above.

Stabilizers such as sugars such as sucrose, anti-oxidants such as alpha-tocopherols, or preservatives that maintain the biological activity or improve chemical stability to prolong the shelf life of the one or more bioactive agents may be added to such compositions. Other excipients can include salts such as sodium chloride (NaCl) and calcium chloride, disodium phosphate dehydrate, potassium dihydrogen phosphate.

In an embodiment, to formulate a composition of the invention, a homogenous mixture of S100 lecithin and cholesterol (e.g. 10:1 w:w) are hydrated in the presence of one or more bioactive agents (such as αDC cocktail, TLR3 agonist Poly(I:C) and IFNα, and TLR9 agonists CpG-ODNS and OX-40 agonist), optionally in phosphate buffer, to form liposomes with encapsulated one or more bioactive agents. The liposome preparation may then be extruded, optionally through a manual mini-extruder, and optionally mixed with an additional therapeutic agent or an imaging agent. This suspension may then be lyophilized and reconstituted in a carrier comprising a continuous phase of a hydrophobic substance to form a water-free liposome suspension.

In some embodiments, the composition may be formulated by hydrating a homogenous mixture of at least one lipid component selected from the group consisting of triolein, dipalmytoilphospatidylglycerol (DPPG), hydrogenated soy phospatidylcholine (HSPC), di stearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin (0.01 mg/ml to 0.2 mg/ml), cephalin (0.005 mg/ml to 0.05 mg/ml), sphingomyelin, egg phosphatidylcholine (EPC), disodium succinate hexahydrate (DSH), dimyristoylphosphatidylcholine (DMPC), dimyristoylphosphatidylglycerol (DMPG) (e.g., 10:1 w:w) with one or more bioactive agents to form liposomes encapsulated with a repolarizing agent, a blockading agent or both.

In other embodiments, the composition may be formulated by hydrating a homogenous mixture of at least two of the lipid components selected from the group consisting of triolein, dipalmytoilphospatidylglycerol (DPPG), hydrogenated soy phospatidylcholine (HSPC), di stearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin (0.01 mg/ml to 0.2 mg/ml), cephalin (0.005 mg/ml to 0.05 mg/ml), sphingomyelin, egg phosphatidylcholine (EPC), disodium succinate hexahydrate (DSH), dimyristoylphosphatidylcholine (DMPC), dimyristoylphosphatidylglycerol (DMPG) (e.g., 10:1 w:w) in the presence of one or more bioactive agents, optionally in phosphate buffer, to form liposomes encapsulated with one or more bioactive agents.

In some embodiments, the composition may be formulated by hydrating a homogenous mixture of dioleoyl-phosphatidylcholine (DOPC) and cholesterol (e.g. 10:1 w:w) in the presence of one or more bioactive agents, optionally in phosphate buffer, to form liposomes encapsulated with the one or more bioactive agents.

In an aspect, the present disclosure provides that the compositions comprising one or more bioactive agents (and/or repolarizing agent or blockading agent) is formulated as a nanoparticle. In some embodiments, such as nanoparticle may be a lipid nanoparticle (such as a modified liposome). Modified liposomes at the nanoscale have been shown to have excellent pharmacokinetic profiles for the delivery of DNA, antisense oligonucleotide, siRNA, proteins and chemotherapeutic agents. Accordingly, in some embodiments, the one or more bioactive agents (and/or repolarizing agent or blockading agent) are comprised in lipid nanoparticles. In other embodiments, the one or more bioactive agents are loaded on the surface of the nanoparticles (e.g., lipid particles such as lipid-coated nanoparticles, as shown in Example 1) or liposomes.

Other suitable nanoparticles include polymeric nanoparticles (for e.g. fabricated using biodegradable synthetic polymers, such as polylactide-polyglycolide copolymers, polyacrylates and polycaprolactones, or natural polymers, such as albumin, gelatin, alginate, collagen and chitosan), polymeric micelles, hydrogel nanoparticles, dendrimers, noble metal nanoparticles (such as gold nanoparticles), and the like.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the release of one or more bioactive agents from nanoparticle- and liposome-responsive polymers, or hydrogel, may be triggered by a change in pH, temperature, radiofrequency or magnetic field.

In an aspect, the present disclosure provides that the liposomes, microparticles, microspheres, nanocapsules, nanoparticles, nanospheres, lipid particles, vesicles, and micelles are formulated for target-specific drug delivery. Such target specificity is effected by conjugating the nanoparticles and liposomes with one or more cell targeting agents that targets APCs, such as DCs, M1-macrophages, and B-cells. The cell targeting agents suitable for the purpose of this invention can be any APC-specific cell surface molecule. Where repolarization of M2-DCs to immunogenic DC1s is desired, the cell targeting agents can be any M2-DC-selective cell surface molecule. The present disclosure provides that nanoparticles and liposomes are coated with one or more of M2-DC-selective cell surface molecule. One of skill in the art will recognize that M2-DC-selective cell surface molecules may be present exclusively on M2-DC-but may also be present on other DCs. However, suitable M2-DC-selective cell surface molecules are those that are expressed exclusively on M2-DCs or expressed at a higher level on M2-DCs than on non-M2-DCs. M2-DC-selective cell surface molecule suitable for the purpose of this invention include, without limitation, C1q protein. Other cell targeting agents suitable for the present disclosure include, but are not limited to, cell surface molecules on APCs, such as DEC-205, Clec9A (DNGR-1), DC-SIGN, BDCA1, BDCA2, BDCA3 and BDCA4.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

Nanoparticles may be prepared by any of the methods known in the art (see Pal et al., Journal of Applied Pharmaceutical Science 01 (06); 2011: 228-234; Hu Y et al. Nanoparticles. Nano Letters. 2007; 7:3056-3064; Hu Y H et al. Biomacromolecules. 2009; 10:756-765; Lynn D M, Langer R. Journal of the American Chemical Society. 2000; 122:10761-10768).

Addition of the one or more bioactive agents, polarizing agents, blockading agent, and additional therapeutic agent may be effected by any suitable means known. By way of non-limiting examples, DNA sequences, linear or as plasmids and cassettes may added to the liposomes, microparticles, microspheres, nanocapsules, nanoparticles, nanospheres, lipid particles, vesicles, and micelles as described by Goncalves et al, Su et al.

The present disclosure provides that the lipid to nucleic acid ratio (LNR) of liposomes, microparticles, microspheres, nanocapsules, nanoparticles, nanospheres, lipid particles, vesicles, and micelles ranges from 30:1 to 2.5:1. In some embodiments, the LNR of liposomes, microparticles, microspheres, nanocapsules, nanoparticles, nanospheres, lipid particles, vesicles, and micelles ranges from 25:1 to 5:1. In at least one embodiment the LNR of liposomes, microparticles, microspheres, nanocapsules, nanoparticles, nanospheres, lipid particles, vesicles, and micelles ranges from 20:1 to 10:1.

The present disclosure provides that the lipid to protein ratio (LPR) of liposomes, microparticles, microspheres, nanocapsules, nanoparticles, nanospheres, lipid particles, vesicles, and micelles ranges from 30:1 to 1:1. In some embodiments, the LNR of liposomes, microparticles, microspheres, nanocapsules, nanoparticles, nanospheres, lipid particles, vesicles, and micelles ranges from 25:1 to 5:1. In at least one embodiment the LNR of liposomes, microparticles, microspheres, nanocapsules, nanoparticles, nanospheres, lipid particles, vesicles, and micelles ranges from 20:1 to 10:1.

The present disclosure provides that the small molecule drug to lipid (D/L) ratio of liposomes, microparticles, microspheres, nanocapsules, nanoparticles, nanospheres, lipid particles, vesicles, and micelles of the present disclosure typically is at least 0.05, 0.1, 0.2, 0.35, 0.5, or at least 0.65 mg of the drug per mg of lipid. In terms of molar ratio, the D/L ratio according to the present disclosure is at least from about 0.02, to about 5, from at least 0.1 to about 2, and from about 0.15 to about 1.5 moles of the drug per mole of the lipid.

In an aspect, the present disclosure provides that the compositions comprising one or more bioactive agents further comprises an imaging agent, a dye or a contrasting agent. Suitable imaging agents can be gadolinium chelates, superparamagnetic iron oxide nanoparticles (SPION), 19F perfluorocarbon nanoparticles, and other magnetic reporter genes, such as metalloprotein-based MM probes. Addition of imaging agents, dyes and contrasting agents disclosed herein provide an added advantage of tracking the intraductal delivery of the compositions disclosed herein and migration of the APCs that phagocytose, pinocytose or take up by other mechanism these compositions.

In some embodiments, the compositions are formulated as depot formulations, extended-release formulations, controlled release formulations.

Compositions disclosed herein are suitable for intraductal administration to a breast duct of a subject.

In an aspect, the present disclosure provides that compositions disclosed herein may be delivered intraductally into one or more breast ducts of a subject by any of the methods known in the art. These include, but are not limited to, injections using syringe/needle (Krause et al. J. Vis. Exp. 2013; (80): 50692), microneedles (for example, solid-, drug-coated-, hollow- and dissolving-microneedles), cannula(e), microcannula(e), catheters, microcatheters, and probes.

Microneedles that are suitable for transdermal administration of drugs can be used for the practice of the present disclosure (Kim et al. Adv Drug Deliv Rev. 2012 November; 64(14): 1547-1568, incorporated in its entirety).

As a non-limiting example, in some embodiments, compositions disclosed herein may be intraductally administered to a breast milk duct of the subject comprising contacting a composition disclosed herein, contained within a treatment chamber of a device with a nipple of a breast and applying positive pressure on the composition such that the composition is forced into the breast milk via the milk duct orifice duct due to the positive pressure. Preferably, the composition is forced into one or more breast ducts. In other embodiments, the composition is forced into 2 to 5 breast ducts, into 4 to 8 breast ducts, or into 7 to 11 breast ducts.

For example, U.S. Pat. No. 6,413,228 discloses a ductal access device that is capable of collecting ductal fluid and infusing the ductal with wash fluid. Such a device can be adapted or configured appropriately for the purpose of this disclosure for the intraductal delivery of compositions of the present disclosure.

As an alternate method of intraductal administration, a small pump may be installed in the duct or at the surface of the nipple with access to the duct for slow continuous administration of the composition to the ductal region e.g., a pump may be installed in the lactiferous sinus for administering the compositions therein and causing a diffusion of the composition to the rest of the duct or the pump may be installed on the nipple surface with access to the duct. A pump installed at the nipple surface can be shaped e.g., like a tack (or a thimble-shaped portion having a top or tack portion and the rest on the nipple surface with a portion extending into a duct requiring treatment or having a risk of requiring treatment. The pump mechanism can comprise e.g. a Duros™ osmostic (micro)pump (Viadur), manufactured by Alza Corp acquired by Johnson & Johnson, IntelliDrug, Alzet® (Durect Corp.), Ivomec SR® bolus etc. (Herrlich et al. Advanced Drug Delivery Reviews. 2012, pages 1617-1626).

Osmotic pumps may also be assembled or configured essentially as the pumps described in U.S. Pat. Nos. 5,531,736, 5,279,608, 5,562,654, 5,827,538, 5,798,119, 5,795,591, 4,552,561, or U.S. Pat. No. 5,492,534, with appropriate modifications in size and volume for administration to the duct of a breast, e.g. for placement into the duct (e.g. the lactiferous sinus) or for placement on the nipple surface. The tip (that accesses the duct) may be able to rotate in order to accommodate ducts of various positions on the nipple surface. A single tack-head pump can have one or more tips placed below the tack-head in order to access a particular duct or ducts, e.g. where two or more ducts in a breast need to be accessed. The pump so configured and loaded with an appropriately formulated compositions disclosed herein for intraductal administration, may administer the compositions as described, but may also contain and administer agents other than the compositions disclosed herein for an appropriate therapeutic purpose for treatment of a precancer or cancer condition in a breast duct. Conceivably the pump may be configured to administer to all the ducts located in the breast, with some size and volume alterations.

In another aspect, intraductal delivery of compositions disclosed herein may be aided with iontophoresis which involves application of an electric current to the breast which aid the migration of the cells into and/or within a duct of the breast.

The timing and size of dosing of compositions disclosed herein are generally designed to reduce risk of or minimize toxic outcomes and/or improve efficacy, such as providing faster and increased exposure of the subject to the compositions, e.g., over time. The quantity and frequency of administration will be determined by such factors as the condition of the patient, age, weight, tumor size and stage, surface area, and severity of the subject's disease, although the appropriate dosage may be determined by attending physician.

Optimal dosages and dosing regimen can be readily determined by a person of skill in the art of medicine by monitoring the patient for signs of disease and adjusting the treatment accordingly. Compositions can be administered multiple times at these dosages. Accordingly, the methods can involve a single dose or multiple doses over a period of time or continuous dose for e.g. by infusion. In some embodiments, a dose can be a single unit dose. In other embodiments, a single dose can be a split unit dose. As used herein, the term “split unit dose” refers to a unit dose that is split so that it is administered over more than one time during a day, including over more than one day. As split unit dose for the purpose of this invention is considered a single i.e., one unit dose. Exemplary methods of splitting a dose include administering 25% of the dose the first day and administering the remaining the next day. In another embodiment, the unit dose may be split into 2, 50% each to be delivered on 2 consecutive days. In yet another embodiment, a split unit dose may be given on 2 alternate days. In still another embodiment, the unit dose may be split into 3 to be administered equally on 3 consecutive days.

In an aspect, the present disclosure provides that the compositions comprising any of liposomes, microparticles, microspheres, nanocapsules, nanoparticles, nanospheres, lipid particles, and vesicles are administered intraductally at a concentration of 1×10⁴ to 1×10⁸ liposomes microparticles, microspheres, nanocapsules, nanoparticles, nanospheres, lipid particles, or vesicles per unit dose.

In an aspect, the present provides that the subjects are dosed with composition disclosed herein wherein the one or more bioactive agents are intraductally administered in dose ranges as provided in Table 2.

TABLE 2
Exemplary Bioactive Dose Ranges.

| No. | Bioactive Agent | Exemplary Dose Range |
|---|---|---|
| 1 | TNFα | 0.01 μg/m² to 200 μg/m² |
| 2 | IL-1β | 0.2 μg/m² to 20 μg/m² |
| 3 | IFNγ | 1 μg/m² to 100 μg/m² |
| 4 | IFNα-2b | 1 μg/m² to 300 μg/m^{2,} |
| 5 | Poly (I:C) | 0.1 μg/m² to 50 μg/m² |
| 6 | TLR9 Agonist CpG-ODNs | 0.01 mg/m² to 40 mg/m² |
| 7 | OX-40 agonist | 0.01 mg/m² to 500 mg/m² |

In some embodiments, the present disclosure provides that subjects are intraductally administered compositions comprising TNFα at unit doses ranging from 0.05 μg/mL to 150 μg/mL, from 0.1 μg/mL to 100 μg/mL, and from 0.5 μg/mL to 50 μg/mL. In some embodiments, the compositions comprise TNFα are administered at a unit dose of 0.1 μg/mL, 0.5 μg/mL, 1 μg/mL, 5 μg/mL, 10 μg/mL, 20 μg/mL, 25 μg/mL, 50 μg/mL, 100 μg/mL, 150 μg/mL and 200 μg/mL.

In some embodiments, the present disclosure provides that subjects are intraductally administered compositions comprising IL-1β at unit doses ranging from 0.01 μg/mL to 20 μg/mL, from 0.1 μg/mL to 15 μg/mL, from 0.5 μg/mL to 10 μg/mL, and from 1 μg/mL to 10 μg/mL per unit dose. In some embodiments, the compositions comprise IL-1β at unit dose of 0.1 μg/mL, 0.5 μg/mL, 1 μg/mL, 5 μg/mL, 10 μg/mL, and 20 μg/mL.

In some embodiments, the present disclosure provides that subjects are intraductally administered compositions comprising IFNγ in unit doses ranging from 1 μg/mL to 100 μg/mL, from 10 μg/mL to 80 μg/mL, from 25 μg/mL to 75 μg/mL, and from 50 μg/mL to 75 μg/mL. In some embodiments, the compositions disclosed herein comprise IFNγ at unit dose of 1 μg/mL, 10 μg/mL, 15 μg/mL, 20 μg/mL, 25 μg/mL, 50 μg/mL, 75 μg/mL and 100 μg/mL.

In some embodiments, the present disclosure provides that subjects are intraductally administered compositions comprising IFNα in unit doses ranging from 1 μg/mL to 300 μg/mL, from 10 μg/mL to 250 μg/mL, from 25 μg/mL to 200 μg/mL, and from 50 μg/mL to 150 μg/mL. In some embodiments, the compositions disclosed herein comprise IFNα at unit dose of 1 μg/mL, 10 μg/mL, 15 μg/mL, 20 μg/mL, 25 μg/mL, 50 μg/mL, 75 μg/mL, 100 μg/mL, 125 μg/mL, 150 μg/mL, 200 μg/mL, 250 μg/mL and 300 μg/mL.

In some embodiments, the present disclosure provides that subjects are intraductally administered compositions comprising TLR3 agonists such as Poly (I:C) in unit doses ranging from 0.01 μg/mL to 50 μg/mL, from 0.1 μg/mL to 40 μg/mL, from 0.5 μg/mL to 25 μg/mL, and from 1 μg/mL to 20 μg/mL. In some embodiments, the compositions comprise Poly (I:C) at unit dose of 0.01 μg/mL, 0.1 μg/mL, 0.5 μg/mL, 1 μg/mL, 5 μg/mL, 10 μg/mL, 20 μg/mL, 25 μg/mL, 30 μg/mL, 35 μg/mL, 40 μg/mL, 45 μg/mL, and 50 μg/mL.

In some embodiments, the present disclosure provides that subjects are intraductally administered compositions comprising TLR9 agonists such as CpG-ODNs in unit doses ranging from 0.01 μg/mL to 20 mg/mL, from 0.1 μg/mL to 15 mg/mL, from 1 μg/mL to 10 mg/mL, from 10 μg/mL to 5 mg/mL, from 50 μg/mL to 1 mg/mL. In some embodiments, the compositions comprise CpG-ODNs at unit dose of 0.01 μg/mL, 0.1 μg/mL, 0.5 μg/mL, 1 μg/mL, 5 μg/mL, 10 μg/mL, 20 μg/mL, 25 μg/mL, 50 μg/mL, 100 μg/mL, 200 μg/mL, 300 μg/mL, 400 μg/mL, 0.5 mg/mL, 1 mg/mL, 2 mg/mL, 4 mg/mL, 6 mg/mL, 8 mg/mL, 10 mg/mL, 15 mg/mL, and 20 mg/mL.

In some embodiments, the present disclosure provides that subjects are intraductally administered effective amounts of compositions comprising OX-40 agonists such as an anti-OX-40 antibody in unit doses ranging from 0.01 mg/mL to 50 mg/mL, 0.1 mg/mL to 40 mg/mL, 0.5 mg/mL to 30 mg/mL, and 1 mg/mL to 25 mg/mL. In some embodiments, the compositions comprise OX-40 agonists at unit dose of 0.01 mg/mL, 0.05 mg/mL, 0.1 mg/mL, 0.5 mg/mL, 1 mg/mL, 2 mg/mL, 4 mg/mL, 6 mg/mL, 8 mg/mL, 10 mg/mL, 15 mg/mL, 20 mg/mL, 25 mg/mL, 30 mg/mL, 35 mg/mL, 40 mg/mL, 45 mg/mL and 50 mg/mL.

In some embodiments, the present disclosure provides that subjects are intraductally administered effective amounts of compositions comprising αDC cocktail (TNFα, IL-1β, IFNγ, IFNα, and Poly (I:C)). In some embodiments, the present disclosure provides that subjects are intraductally administered compositions comprising the αDC cocktail, wherein a unit dose of the composition comprises: TNFα ranging from 0.05 μg/mL to 150 μg/mL, from 0.1 μg/mL to 100 μg/mL, and from 0.5 μg/mL to 50 μg/mL; IL-1β ranging from 0.01 μg/mL to 20 μg/mL, from 0.1 μg/mL to 15 μg/mL, from 0.5 μg/mL to 10 μg/mL, and from 1 μg/mL to 10 μg/mL; IFNγ ranging from 1 μg/mL to 100 μg/mL, from 10 μg/mL to 80 μg/mL, from 25 μg/mL to 75 μg/mL, and from 50 μg/mL to 75 μg/mL; from 1 μg/mL to 300 μg/mL, from 10 μg/mL to 250 μg/mL, from 25 μg/mL to 200 μg/mL, and from 50 μg/mL to 150 μg/mL; and Poly (I:C) ranging from 0.01 μg/mL to 50 μg/mL, from 0.1 μg/mL to 40 μg/mL, from 0.5 μg/mL to 25 μg/mL, and from 1 μg/mL to 20 μg/mL.

In some embodiments, the present disclosure provides that subjects are intraductally administered compositions comprising a TLR9 agonist CpG-ODNs and an OX-40 agonist antibody, wherein a unit dose of the compositions comprises CPG-ODNs ranging from 0.01 μg/mL to 20 mg/mL, from 0.1 μg/mL to 15 mg/mL, from 1 μg/mL to 10 mg/mL, from 10 μg/mL to 5 mg/mL, from 50 μg/mL to 1 mg/mL, and an OX-40 agonist antibody ranging from 0.01 mg/mL to 50 mg/mL, 0.1 mg/mL to 40 mg/mL, 0.5 mg/mL to 30 mg/mL, and 1 mg/mL to 25 mg/mL.

In some embodiments, the present disclosure provides that subjects are intraductally administered compositions comprising a TLR3 agonist Poly (I:C) and IFNα, wherein a unit dose of the composition comprises Poly (I:C) ranging from 0.01 μg/mL to 50 μg/mL, from 0.1 μg/mL to 40 μg/mL, from 0.5 μg/mL to 25 μg/mL, and from 1 μg/mL to 20 μg/mL, and IFNα ranging from 1 μg/mL to 300 μg/mL, from 10 μg/mL to 250 μg/mL, from 25 μg/mL to 200 μg/mL, and from 50 μg/mL to 150 μg/mL.

Methods disclosed herein involve administering one or more consecutive doses of compositions into a breast duct of a subject who may have received a first dose, and/or administering the first and one or more subsequent doses. The doses are administered in particular amounts and according to particular timing schedule and parameters.

In another aspect, the first dose is administered intraductally and any subsequent dose is administered by any suitable means, including intraductally, by injection and by infusion, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In some embodiments, the methods generally involve administering intraductally the first dose of compositions disclosed herein thereby reducing the disease burden. This may be followed by a subsequent dose of composition administered during a particular time of window with respect to the first dose or the administration of the subsequent dose to a subject having received a first dose. The first dose in some embodiments is relatively low. The amount of compositions administered and the timing of the doses of compositions are designed to improve one or more outcomes, such as maturation of APCs (breast tissue resident, tumor tissue resident or inbound APCs), increased migration of fully mature antigen-loaded APCs to draining lymph nodes (and if present to ectopic and/or tertiary lymph nodes), activation of any of the effector cell functions of T-cells, B-cells, and/or NK cells, increased breast tumor infiltration by any of T-cells, B-cells and/or NK cells, reduction in T-regs, reduction in the immunosuppression, reduction in tumor size and increase in pro-inflammatory cytokines, chemokines, repolarization of M2-DCs, reduction or prevention of M2-shift, recruitment of cytotoxic T-lymphocytes, and other immune response.

In some embodiments, disclosed herein are methods involving administration of subsequent doses of composition at an increased number, and thus a higher dose, than the first/initial dose.

Where dosing regimen involves multiple doses, each dose may be administered daily, several times a day (twice, thrice, four times and the like), alternate days, every 2 days, 3 days, 5 days, 7 days, 14 days, 15 days, every 3 weeks, 28 days, monthly, quarterly, 6 monthly, and annually.

In some aspects, the timing of doses following initial dose is measured from the initiation of the initial (first) dose to the initiation of the next dose. In other embodiments, the timing of doses following initial dose is measured from the completion of the initial (first) dose.

The present disclosure encompasses the initial dose may be a split unit dose followed by a second dose administered thereafter. In some embodiments, a second or a subsequent dose may be a split unit dose. By way of a non-limiting example, a split unit dose may be administered over three days and the second unit dose is administered the very next day or it may be administered a year later. Initial dose is intended to create any limitations with regards to a subject in need of such a dose by imply that the subject has never before received a dose of cell therapy or even that the subject has not before received a dose of the same cells expressing the same recombinant receptor or targeting the same antigen.

Generally, compositions as described herein may be administered intraductally 1×10⁴ to 1×10⁸ of any of liposomes, microparticles, microspheres, nanocapsules, nanoparticles, nanospheres, lipid particles, or vesicles per unit dose.

### Combination Therapy

The present disclosure contemplates that intraductal methods disclosed herein further comprise combination therapy. In an aspect, the intraductal method further comprises administering to the subject one or more additional therapeutic agent or therapy. The additional therapeutic agents may be administered to the subject by any suitable means known in the art, including without limitation, intraductally, topically, orally, nasally, parenterally by injection or infusion, subcutaneously, etc. The additional therapeutic agents may be comprised in the compositions disclosed herein or may be independently formulated. The order of administration of the therapeutic agents and/or therapy may be in any order of administration. For example, the compositions disclosed herein may be co-administered with an additional therapeutic agent or the additional therapeutic agent may be administered first or the additional therapeutic agent may be administered after compositions of the present disclosure are administered.

The additional therapeutic agents can be any that is useful for the purpose of this invention.

Exemplary additional therapeutic agents include, without limitation, checkpoint inhibitors, anti-hormonals directed to reducing estrogen in subjects with breast cancer (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubincin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubincin, idarubicin, mitoxantrone, oxaliplatin, paclitaxel, bleomycin, bortezomib, oncolytic viruses, patupilone, Tyrphostin AG 490 (JAK2/STAT3 inhibitor), DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inibitors, anti-TGF-β inhibitors, checkpoint inhibitors (such as PD-1 antibodies, anti-PD-1L antibodies, anti-PD-L2 antibodies, anti-CTLA-4 antibodies, anti-LAG-3 antibodies, anti-TIM-3 antibodies, and the like), anti-CCR4 inhibitors, anti-FoxP3 inhibitors, cell therapy such as Chimeric Antigen Receptor/T-cell (CAR-T) therapies, and other adoptive cell therapies, or a combination thereof.

Additional therapy may include surgery, radiation, chemotherapy, acupuncture, adoptive cell therapy, etc. The methods disclosed herein may be used as a primary therapy, neoadjuvant therapy (for example, before surgery (such as mastectomy or lumpectomy) or chemotherapy), or adjuvant therapy (for illustrative purposes only, after chemotherapy or treatment with other methods of adoptive cell therapy).

### Articles of Manufacture

Also provided herein are articles of manufacture such as kits and devices, for the administration of the cells and compositions disclosed herein for adoptive cell therapy, and for storage and administration of the cells and compositions.

The articles of manufacture include a composition disclosed herein, one or more containers, packaging material, and a label or package insert generally including instructions for administration of the cells to a subject.

The containers contain one or more unit doses of the composition to be administered. In some embodiments, the article of manufacture comprises one or more containers, each containing a single unit dose of the compositions. The unit dose may be an amount or number of any of: liposomes, microparticles, microspheres, nanocapsules, nanoparticles, nanospheres, lipid particles, and vesicles to be administered to the subject in the first dose or twice the number (or more) the nanoparticles, liposomes, and the like to be administered in the first or subsequent dose(s). It may be the lowest dose or lowest possible dose of the nanoparticles, liposomes and the like that would be administered to the subject in connection with the administration method.

In some embodiments, each of the containers individually comprises a unit dose of the composition that contains the same or substantially the same number of any of: liposomes, microparticles, microspheres, nanocapsules, nanoparticles, nanospheres, lipid particles, and vesicles. Thus, in some embodiments each of the containers comprises the same or approximately or substantially the same number of any of: liposomes, microparticles, microspheres, nanocapsules, nanoparticles, nanospheres, lipid particles, and vesicles. In some embodiments, the unit dose includes 1×10⁴ or more of any of: liposomes, microparticles, microspheres, nanocapsules, nanoparticles, nanospheres, lipid particles, and vesicles. In some embodiments, the unit dose includes 1×10⁴ to 1×10⁸ of any of: liposomes, microparticles, microspheres, nanocapsules, nanoparticles, nanospheres, lipid particles, and vesicles.

In some embodiments, the articles of manufacture further include an additional therapeutic agent such as checkpoint inhibitors, anti-hormonals directed to reducing estrogen in subjects with breast cancer (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, thymidylate-targeted drugs (such as docetaxel, gemcitabine, paclitaxel or carboplatin and pegylated liposomal doxorubicin), cytotoxic agents such as alkylating agents (such as temozolomide and cyclophosphamide), anthracyclines (such as doxorubicin, pegylated liposomal doxorubicin, epirubincin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubincin, idarubicin, mitoxantrone, oxaliplatin, paclitaxel, bleomycin, bortezomib, oncolytic viruses, patupilone, Tyrphostin AG 490, a Janus Activated Kinase 2/signal trasducer and activator of transcription-3 (JAK2/STAT3) inhibitor, DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inibitors, anti-TGF-β inhibitors, trastuzumab, ado-trastuzumab emtansine, pertuzumab, abemaciclib, palbociclib, cell therapy such as Chimeric Antigen Receptor/T-cell (CAR-T) therapies, and other adoptive cell therapies. Anti-hormonals can be selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652. Anti-checkpoint inhbitors can be selected from the group consisting of anti-PD-1, anti-PD-1L, anti-PD-L2, anti-CTLA-4, anti-LAG-3, anti-TIM-3, anti-OX40, and the like.

In some embodiments, the articles of manufacture further include an imaging agent, a dye or a contrasting agent selected from the groups consisting of gadolinium chelates, superparamagnetic iron oxide nanoparticles (SPION), 19F perfluorocarbon nanoparticles.

Suitable containers include, without limitation, for example, bottles, vials, syringes, and flexible bags, such as infusion bags. The containers may be formed from a variety of materials such as glass or plastic. In some embodiments, the container has one or more port, e.g., sterile access ports, for example, for connection of tubing or cannulation to one or more tubes, e.g., for transpapillary delivery and/or for connection for purposes of transfer to and from other containers, such as cell culture and/or storage bags or other containers.

The article of manufacture may further include a package insert or label with one or more pieces of identifying information and/or instructions for use. In some embodiments, the information or instructions indicates that the contents can or should be used to treat a breast disorder and/or providing instructions therefor. The label or package insert may indicate that the contents of the article of manufacture are to be used for treating the breast disorder. In some embodiments, the label or package insert provides instructions to treat a subject, e.g., the subject having a breast disorder, via a method involving the intraductal administration of a first and one or more subsequent doses of the cells, e.g., according to any of the embodiments of the provided methods. In some embodiments, the instructions specify administration, in a first dose, of one unit dose, e.g., the contents of a single individual container in the article of manufacture, followed by one or more subsequent doses at a specified time point or within a specified time window and/or after the detection of the presence or absence or amount or degree of one or more factors or outcomes in the subject.

As used herein, the terms “a,” “an,” and “the” include plural reference unless the context dictates otherwise.

As used herein, the term “about” refers to a measurable value such as an amount, a temporal duration and the like, is meant to encompass variations of ±20% or in some instances ±10%, or in some instances ±5%, in some instances ±1%, and in some instances ±0.1% from the specified value, as such variations are appropriate to perform the disclosed methods.

As used herein, the term “activation” refers to the state of a cell that has been sufficiently stimulated to induce detectable cellular proliferation. Activation can also be associated with induced cytokine production, and detectable effector functions. The term “activated T-cells” refers to, among other things, T-cells that are undergoing cell division.

As used herein, the term “adjuvant therapy” refers to a therapy that follows a primary therapy and that is administered to subjects at risk of relapsing. These are subjects who have a history of breast disorder and have been treated with another mode of therapy. Adjuvant systemic therapy in case of breast cancer usually begins soon after primary therapy to delay recurrence, prolong survival or cure a subject. As used herein “primary therapy” refers to a first line of treatment upon initial diagnosis of a breast disorder in a subject. Non-limiting exemplary primary therapies may involve surgery, a wide range of chemotherapies, and radiotherapy.

As used herein, the term “antibody,” refers to an immunoglobulin molecule which specifically binds with an antigen. Antibodies can be polyclonal or monoclonal, multiple or single chain, or intact immunoglobulins, and may be derived from natural sources or from recombinant sources. Antibodies such as IgG are typically tetramers of immunoglobulin molecules.

As used herein, the term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv, linear antibodies, single domain antibodies such as sdAb (either VL or VH), camelid VHH domains, and multispecific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and is capable of being expressed as a single chain polypeptide, and wherein the scFv retains the specificity of the intact antibody from which it is derived. Unless specified, as used herein an scFv may have the VL and VH variable regions in either order, e.g., with respect to the N terminal and C-terminal ends of the polypeptide, the scFv may comprise VL-linker-VH or may comprise VH-linker-VL.

As used herein, the terms “subject,” “patient,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

As used herein, a “route of administration” or “route of delivery” refers to a pathway for delivering the compositions to a subject, typically referring to a location where the composition is administered (for example, oral, intravenous, and the like) or a target of the action is (for example, topical, enteral, parenteral and the like). “Route of administration” or “route of delivery” may be used interchangeably in the present disclosure.

### EXAMPLES

### Example 1

### Synthesis of Lipid-Coated CpG-ODN and CpG-ODN+OX-40 Agonist Antibody Nanoparticles

Preparation of Lipid-coated Nanoparticles. Lipid-coated nanoparticles with a poly-1 core will be synthesized as described by Su et al. (Molecular Pharmaceutics 8, no. 3 (Jun. 6, 2011): 774-787). Briefly, lipid-coated nanoparticles with a poly-1 core will be synthesized via two different processes: a double emulsion/solvent evaporation approach or a solvent diffusion/nanoprecipitation strategy. For double emulsion synthesis, 30 mg of poly-1 (or PLGA for pH-insensitive control particles) and 2 mg of the phospholipids DOPC, DOTAP, and DSPE-PEG in a 7:2:1 molar ratio will be co-dissolved in 1 ml of dicholoromethane (DCM). 200 μl Phosphate buffered saline (PBS) will be then added to the mixture on ice during a 1 min sonication step at 7 W using a probe tip sonicator (Misonix XL2000, Farmingdale, N.Y.) to form a first emulsion. The primary emulsion will be then dispersed into 6 ml of distilled, deionized nuclease-free water with sonication at 12 W for 5 min, before leaving on a shaker for 18 h at 25° C. to evaporate the organic solvent. For nanoprecipitation synthesis, the same quantities of DOPC, DOTAP, and polymer will be co-dissolved in 4 ml of ethanol and added drop-wise to 40 ml of distilled, deionized nuclease-free water, followed by gentle stirring for 5 h to evaporate ethanol. DSPE will be introduced into the lipid coating via a post-insertion process: DSPE lipid will be added at 1 mM to 0.5 mg/ml particles in distilled, deionized nuclease-free water and the suspension will be stirred for 16 h at 25° C. The particles will be collected and washed once via centrifugation, resuspended in fresh water and stored at 4° C. until use. Lipid-free PVA stabilized particles will be prepared by similar processes except the organic emulsion or solution containing polymer only will be dispersed into a 2% w/vol PVA aqueous solution.

A fraction of each particle batch will be dried in a vacuum oven to determine the particle concentration (mg/ml) by measuring the dry mass. Dynamic light scattering (DLS) and zeta potential measurements will be used to determine the particle size and surface charge using a ZetaPALS dynamic light scattering detector (Brookhaven Instruments). To estimate the percentage of lipid incorporated and the resultant mol % of lipid present in the lipid surface coating, lipid-enveloped particles will be prepared by nanoprecipitation as described above with 1 mol % DOPE-rhodamine added as a tracer for measuring the total amount of lipid incorporated into the particles, before the post-insertion of a DSPE-lipid labeled with carboxyfluorescein (DSPE-CF). The lipids will be then stripped from the particle surface by treatment with 2% triton X-100 for 15 min and the supernatant will be measured for both rhodamine and fluorescein signals to quantitate the amount of lipid incorporated.

To investigate the surface structure of lipid-coated nanoparticles by cryoelectron microscopy (cryoEM), particles will be embedded in ice by blotting a particle suspension (3 uL) on a 1.2/1.3 μm holey carbon-coated copper grid (Electron Microscopy Sciences) and immediately freezing the sample in liquid ethane using a Leica plunge-freezing machine. Samples will be transferred to a cryogenic holder and imaged using a JEOL 2200FS transmission electron microscope at 185 μA emission current and 40 000× magnification.

CpG-ODN Loading. CpG-ODNs available from Invivogen, USA. (1.5 mL, 200 μg/mL, pH=5.7) will be then coated onto the nanoparticles. 1 to 3 layers will be deposited. After the assembly, the supernatant will be collected by centrifugation (15000 rpm, 25 min). The amount of CpG in the supernatant will be character by UV-Vis spectroscopy. The absorbance at 262 nm and 278 nm will be used to characterize CpG. The amount of CpG loaded onto the particles will be calculated by subtracting the amount of CpG in the supernatant compared with the original materials added.

OX-40 agonist antibody loading. For CpG-ODN nanoparticles also loaded with OX-agonist antibody, the OX-40 agonist antibody will be deposited onto the surface of the nanoparticle (2 mL, 200 mg/mL) will be used for coating on to the nanoparticle. 1 to 3 layers will be deposited The amount of antibody will be determined by subtracting the amount of antibody in the supernatant compared with the original materials added.

### Example 2

### Synthesis of α-Type 1 Polarizing Nanoparticles

Nanoparticles encapsulating α-type-1 polarizing bioactive agents (αDC Cocktail) will be prepared according to the methods described by Kocbek et al., J. Control. Release, 120, 18-26 (2007). Clinical grade Poly (I:C) can be purchased as Rintatolimod or Ampligen® commercially from Hemispherx Pharmaceuticals.

Briefly, 400 μL of αDC cocktail (TNFα (500 μg/mL), IL-1β (2.5 μg), IFNγ (1250 μg/mL), PEG-IFNα-2b (1250 μg/mL), and Poly I:C) (100 μg/mL)) will be added to ethyl acetate containing 200 mg of poly(lactic-co-glycolic acid) (PLGA, Sigma-Aldrich). The mixture will be then stirred at 20,000 rpm (Homogeniser, IKA) with simultaneous sonication (Ultrasonic bath: 500 W, 30 kHz, BRANSON). After 3 min of emulsification, 8 mL of aqueous solution of polyvinyl alcohol (PVA, 5%) will be added to a water/oil emulsion to form a water/oil/water double emulsion and this will be stirred for 5 min. To solidify the nanoparticles, the organic solvent will be evaporated by stirring the double emulsion with 200 mL of aqueous solution of 0.1% PVA at 500 rpm for 2 h. For complete removal of ethyl acetate, the dispersion of nanoparticles will be concentrated to approximately half the volume using a rotary evaporator at 40° C. The resulting nanoparticles will be centrifuged at 3,000 rpm for 20 min, and washed twice with phosphate-buffered saline (PBS). The mean size of the particles will be measured using a particle size analyzer (ELS-Z, Otsuka).

For some batches, nanoparticles comprising bioactive agents IFNα-2b, Poly (I:C), TNFα, IL-1β, IFNγ, PEG-IFNα, and Poly I:C will be prepared individually using the method described in this example. The nanoparticles comprising the individual bioactive agents will be combined to create a cocktail of the bioactive agents.

The concentration of the bioactive agents will be determined after lysing the nanoparticles in a lysis buffer containing 0.1% SDS and 0.1 N NaOH. The amounts of TNFα, IL-1β, IFNγ, PEG-IFNα-2b, and Poly I:C will be determined at 260° nm.

### Example 3

### Intraductal Administration of Bioactive Agents TLR9 Agonist CpG-ODNs and OX-40 Agonist

Treatment Protocol. The primary objective of the study is to evaluate safety and tolerability of intraductal administrations of TLR9 agonist CpG-ODNs and OX-40 agonist (for example, MOXR0916) (collectively, the “Drugs”) as compared to a placebo. The secondary object of the study is to evaluate (i) mobilization of immune cells, antigen presentation in the subject, and (ii) preliminary efficacy by assessing, overall response rate and progression-free survival after treatment.

Eighty eight (88) subjects with breast cancer will be randomized to receive either treatment with placebo or the Drugs. All subjects will receive radiation therapy on days 1-2. On days 2, 9, 16, and 23, subjects will be administered intraductally (as described below) placebo (Cohort 1; n=8) or 1 mg (Cohort 2; n=20), 5 mg (Cohort 3; n=20), and 10 mg (Cohort 4; n=20) and 20 mg (Cohort 5; n=20) mgs of TLR9 agonist CpG-ODNs. On days 3, 10, 30, 60, 90, 120, and 160, the subjects in Cohorts 2, 3, 4, and 5 will be intraductally administered placebo (n=5) or 10 mg/mL (n=5), 100 mg/mL (n=5) and 200 mgs/mL (n=5) of anti-OX-40 antibody, and subjects in Cohort 1 will receive placebo.

The TLR9 agonist will be a clinical grade Class C CpG-ODN M362 (5′-tcgtcgtcgttc:gaacgacgttgat-3′ (25 mer)) or CpG-ODN 2395 (5′-tcgtcgttttcggcgc:gcgccg-3′ (22 mer)). The OX-40 antibody will be an agonist that mimics OX-40 ligand capable of activating T-cells.

Nipple Preparation. After subjects have disrobed, a clinician will clean the nipple on the breast to be studied. This includes wiping the nipple clean with a slightly granular gel or ointment to loosen and remove any dead skin cells and accumulated oils. This is a cleanser frequently used in hospitals before medical procedures. Afterwards, some numbing cream will be applied to the nipple.

Nipple Anesthetic. 1 mL of Lidocaine mixed with 0.1-0.2 mL of blue dye will be injected with a very small needle into the base of the nipple.

Duct Identification. After dye injection and before the catheter placement, a small, flexible wire will be inserted about ½ inch into the opening to further identify and dilate the duct opening. Once a duct is identified, a small piece of knotted suture material will be inserted into the duct to mark it. This will be done on at least 3 duct openings and as many as 5. If the clinician is unable to find at least 3 duct openings, the subject will be not able to continue in the study and will be withdrawn. These subjects will be replaced by newly enrolled subjects.

Catheter Placement, Instillation of CpG-ODN and anti-OX-40 antibody, and X-ray Examination. Once all of the nipple duct openings are marked, the clinician will insert and place a catheter via the ductal orifice into each marked breast duct so marked. Once the catheters are in place, the clinician may optionally slowly (over 30 seconds) instills less than 1 mL of radio-opaque dye into the ducts to permit imaging of the ducts.

After dye instillation and depending on the number of ducts identified, upto 2 mL (generally ranging from 0.5 mL to 2 mL) of a first composition comprising a Class C CpG-ODN TLR9 agonist and intraductally delivered slowly (over 1 minute) to each duct of the subject on days 2, 9, 16, and 23. A second composition comprising OX-40 agonist antibody with potential immunostimulatory activity will be intraductally administered to each duct of the subject on days 3, 10, 30, 60, 90, 120, and 160. The OX-40 antibody mimics OX-40 ligand to activate the T-cells. Only the breast containing the cancer will be treated. Compositions are intraductally administered into breast ducts in volumes upto 10 mL in batches or sets depending on the number of ducts to be treated.

Attempts will be made to distribute the doses evenly duct-by-duct based upon the number of affected ducts or lobules identified.

The catheter generally will remain in each duct for approximately 1-5 minutes. During the procedure, subjects are asked to assess their pain using a visual analog scale. After dye and nanoparticles have been instilled an image will be taken to demonstrate adequate infusion of modified cells into the ducts. When the number of ducts affected is higher than 2 ducts, the intraductal therapy procedure may be in done in sets. The catheters will be removed from the first set of ducts, for example 2 adjacent duct, and these ducts will be each marked with a small piece of knotted suture material. At this point, subjects will be assessed for pain using the pain scale for pain assessment.

Subsequently, new catheters will be inserted into the remaining marked ducts. After the next half of the nipple ducts have been cannulated, and dye and cells will be infused, another image will be taken with the fluoroscope to document the ducts. Subjects will be asked again to assess their pain. The catheters will be removed and ducts individually marked with a small piece of knotted suture material. Benzoin ointment and a clear plastic dressing (bio-occlusive) will be placed on the nipple to keep the markers in place until surgery. The total procedure takes 0.5 to 1.5 hours. Photographs will be taken of the procedure.

If during assessment of pain, the subject reports Grade 3 or 4 pain in the breast which does not resolve within 10 minutes after infusion of the compositions, study related procedures will be discontinued for that subject. Blood draws and follow-up assessment as well as pathological assessment as described herein will be performed per the protocol. In this case, subjects are replaced in the study group for statistical purposes.

If on initial X-ray examination perforation is noted side effects will be assessed immediately. If the subject does not report any untoward effects, the remaining ducts will be cannulated and administered with nanoparticles. Study related blood draws and assessment as well as pathological assessment as described below will be performed per protocol.

On day 2 of the study dosing, prior to dosing with either placebo or the Drug vital signs (systolic/diastolic blood pressure, pulse, temperature, respiratory rate) and hot flashes occurrence before dosing and at the following time points: 1, 2, 4, 8 and 12 hours following dose administration will be checked. A 12-lead ECG before dosing and at 4 hours following administration of the first dose will be performed. Blood samples for biomarker and pharmacokinetic (PK) analysis will be collected 10±1 minutes prior to dose administration to establish the baseline.

On days 9, 16, 23, 30, 60, 90, 120, 160 and the end of the study, vital signs (systolic/diastolic blood pressure, pulse, temperature, respiratory rate) and hot flashes occurrence will be checked. ECG, within 60 minutes prior to dose administration will be also be performed, hematology, serum chemistry, coagulation and urinalysis and blood samples for PK analysis will be collected 10±1 minutes prior to administration of the study drug.

Biopsies of the tumor will be taken pretreatment and on or after treatment. Serial biopsies of tumor draining lymph nodes will also be taken pretreatment, during treatment and after treatment. Tumor responses will be assessed using RECIST v1.1. Tissues biopsies will be used for immunopheotyping of immune cells and cytokines present in the tissues.

The safety profile and tolerability of each dose group will be determined by clinical assessment, subject reports and active follow-up throughout the study period. All adverse events, regardless of severity, will be recorded and evaluated.

Maturation of APCs and Immune Response. Maturation of the APCs such as pDCs and immune response in a subject will be determined by measuring the release of cytokines such as IL-12p70, IFNα and IL6, increase in maturation markers on APCs, such as expression of CCR7, and co-stimulatory molecules on APCs such as OX-40L, CD40, and increased antigen presentation resulting in activation of effector immune cells such as T-cells, B-cells, and NK cells. Statistical analysis will be performed with Microsoft Excel (Microsoft, Redmond, Wash.) and SPSS v18.0 (SPSS, Chicago, Ill.). Presence of CD11c+HLA-DR+ DCs and CD123+HLA-DR+ DCs (pDC) will be determined and their ratio will be calculated. Data from immune cells and cytokine release will be analyzed with paired t-test. Data from immune cell changes using different groups will be tested with two tailed t-test. Immune cell changes will be analyzed using Pearson's correlation coefficient. P values <0.05 were considered statistically significant.

In vitro Assays—Cytokine release. Subject's serum cytokines levels will be measured to determine changes in the stimulation of immune cells in the subject. Serum cytokine levels of IL6, IL8, MIP-1α, MIP1β, IL10, IL12p70, IL17A, IFNα, IFNγ, TNFα, Il-23, IL-27 and GM-CSF will be assayed before the intraductal delivery of the first composition CpG-ODN at day 2, and after intraductal administration of the composition CpG-ODN at 4 hours, 8 hours, 12 hours, 24 hours, 36 hours, 48 hours, and 72 hours using commercially available ELISA kits such as Human Inflammatory Cytokines Multi-Analyte ELISArray Kit MEH-004A from Qiagen, Germantown, Md. Changes in IL-13 secretion will be determined using a commercially available IL-13 secretion ELISA assay such as those form Miltenyi Biotec and Thermo Fischer or Human IL-13 Luminex Performance Assay from R&D Biosystems. Cytokine secretion will be measured in samples diluted to be in the linear range of the assay. Time-dependent increasing levels of proinflammatory cytokines and chemokines like IL12p70, IL-23, IL-27, IFNα and IL6 will be indicative of activation and maturation of APCs such as pDCs in the subject.

Mobilization of Immune Cells.

APCs. Mature antigen loaded APCs such as DCs, M1-macrophages, and B-cells, are expected to be present in higher levels in breast tissue of Drug-treated subjects compared to controls and are expected to migrate to draining lymph nodes for antigen presentation to effector cells. Thus, mature APCs in draining lymph nodes of Drug-treated subjects are expected to be detected in levels higher than those in placebo subject. Tumor biopsy samples will be tested for presence of matured APCs by immunohistochemistry (IHC) or flow cytometry (according to manufacturer's instructions, e.g., Bio-Rad, AbCam). Tumor biopsy samples will be immunophenotyped or stained with APC maturation makers using such as FITC or PE-labeled antibodies to chemotaxis directing and migration speed regulating molecule CCR7, CD40, CD83, CD86, OX-40L, CD209 and others (available from Bio-RAD, Invitrogen, Biolegend, R&D Systems, AbCam, and BD Biosciences).

Serial lymph node biopsies will be used to determine migration of APCs. DCs expressing CCR7, CD40, CD83, and OX-40L can be detected by immunohistochemistry or flow cytometry on the biopsy tissues. Single cell suspension (SSC) will be prepared from the biopsy lymph node samples. Cell counts and viability will be measured using Improved Neubauer Hemocytometer and trypan blue staining. Samples will be immunophenotyped or stained with APC maturation makers such as FITC or PE-labeled antibodies to CCR7, CD40, CD83, CD86, OX-40L (available from Bio-RAD, Invitrogen, Biolegend, R&D Systems, AbCam, and BD Biosciences).

T-cells. Draining lymph node resident T-cells are expected to be activated by tumor antigen presentation and cytokine signaling by mature antigen loaded APC. Activation of T-cells in draining lymph nodes will be determined by measuring the levels of CD4+ Th1 cells CD8+ cytotoxic T-cells, Tfh cells in lymph node of subjects. Single cell suspension (SSC) will be prepared from the biopsy lymph node samples. Cell counts and viability will be measured using Improved Neubauer Hemocytometer and trypan blue staining. Samples will be immunophenotyped or stained with PE- or FITC-labeled antibodies to CD3, CD4, CD8, CD56, CD19, CD-28, CD69, CD40L, OX-40, CD134, and CD137 (Invitrogen), and fixed with 1% paraformaldehyde prior to being analyzed on LSR II flow cytometer (BD Biosciences, San Jose, Ca) and data will be processed using WinList (Verity Software House, Topsham, Me.).

Infiltration of breast tumor tissue by activated T-cells will be determined using tumor biopsy tissues by IHC or flow cytometry by immunophenotyped or staining with the T-cells markers for CD4+ Th1 cells and CD8+ CTLs as described above.

B-cells, plasma cells, plasmablasts. Infiltration of breast tumor tissue by activated B-cells will be determined using tumor biopsy tissues and subject's peripheral blood mononuclear cells (PBMCs) will be immunophenotyped for B-cells by IHC or flow cytometry as described above by immunophenotyped or staining with PE- or FITC labeled antibodies to B-cell markers (based on Freiburg classification), CD19 allophycocyanin-Cy7, CD20-PE, IgD FITC, IgM PE-Cy5, CD38 PE-Cy7, Igκ allophycocyanin, Igλ, PE, IgG PE-Cy5, CD27 PE, CD21 allophycocyanin, CD40 FITC, CD86 PE-Cy5, early activation marker CD69 FITC (Becton Dickinson), and IgA FITC (Miltenyi Biotec).

Breast tumor tissue of Drug-treated subjects are expected to have greater infiltration of B-cells, and higher numbers of plasma cells and plasmablasts, expressing activation and memory B-cell markers, for example, increase in C80+ B-cells, CD86+ B-cells, CD40L-activated B-cells, and/or CD20+ B-cells (memory cells). Presence of infiltrating B-cells is correlated with positive outcome in subject with breast cancer (Tsou et al. Cancer Research. doi: 10.1158/0008-5472.CAN-16-0431; Mehmoud et al. Breast Cancer Res Treat 2012; 132:545-53).

NK cells. Draining lymph node resident NK cells are expected to be activated by mature antigen loaded APCs and form immune synapses with activated T-cells in higher numbers in Drug-treated subjects compared to placebo-treated subjects. Most lymph node resident NK cells are said to be CD56brightCD16dim/− NK cells (Sta Maria et al. Magn Reson Insights. 2014; 7: 15-2). Activated NK cells are expected to infiltrate breast tumor tissue in Drug-treated subjects in higher numbers compared to placebo treated subjects. Infiltration of breast tumor tissue by activated NK cells will be determined using tumor biopsy tissues and subject's peripheral blood cells will be immunophenotyped for NK cells by IHC or flow cytometry by immunophenotyped or staining with PE- or FITC labeled antibodies to maturation markers such as CD27, CD56, CD57, CD62L, CD94, as well as NKG2D, CD16.

Breast tumors of Drug-treated subjects are expected to have higher numbers of one or more subset of infiltrating cytolytic NK cells, for example, CD57+, CD27high, and CD56dimCD16+ NK cells.

In this study, subject's peripheral blood samples will be analyzed for the levels of CD56dimCD16+ (cytolytic), CD56brightCD16− (immature), and CD56−CD16+, CD56brightCD16+ and CD56dimCD16− (immature) NK cell subsets as described by Mamessier et al. (J Immunol Mar. 1, 2013, 190 (5) 2424-2436). Briefly, NK cells will be negatively isolated from subjects' peripheral blood mononuclear cells (PBMCs) with the NK cell StemSep system (StemCell Technology) according to the manufacturer's instructions. The purity and viability of sorted cells will be established to be >94%. NK cells will be incubated 15 d in RPMI 1640/10% FCS complemented with IL-2 (1000 U/ml; Proleukin; Chiron) and PHA (1/1000; Life Technologies) on irradiated PBMCs used as feeder cells. A third of the media was replaced with fresh RPMI 1640/10% FCS complemented with IL-2 at days 6 and 10.

200 μL of fresh whole blood or 1×10⁶ cells isolated from subject's breast tissues, peripheral blood, and bone marrow will be incubated with the appropriate PE- or FITC labeled antibodies on a rocking platform for 30 min. RBCs will be lysed with OptiLyse B (Beckman Coulter). Samples will be analyzed on a BD FACSCanto (BD Biosciences). Before and after analyzing the samples, fluorescence intensities from the FACSCanto will be standardized over time with photomultiplier tube seven-color setup beads (BD Biosciences) to prevent fluorescence intensity variability related to external or intrinsic factors. The gating strategy consisted in eliminating doublets based on the forward scatter area/forward scatter height parameters, then the dead cells (7-aminoactinomycin D+), then selecting for the CD45+CD3−CD33−HLA-DR−CD56+/− cells.

PBMCs obtained from Drug-treated subjects are expected to have higher levels of cytolytic NK cells (for example, CD56dimCD16+ cytolytic NK cells, higher number of CD57+ NK cells and/or CD27high NK cells) compared to PBMCs obtained from placebo-treated subjects. While about 90% of NK cells in the peripheral blood and spleen are CD56dim CD16+ NK cells, studies have shown that immature CD56brightCD16− and CD56dimCD16− subsets of NK cells are recruited from peripheral blood and are increased in mammary tumors suggesting the immature NK cells are preferentially recruited to breast tumors (Mamessier et al.). This present disclosure's method is expected to activate lymph node resident NK cells, mobilize the autologous cytolytic NK cells from lymph nodes and peripheral blood to the breast tumor tissue and bypass the poor clinical outcome observed with severe side effect in cancer patients, including vascular leak and the logical problems associated with ex vivo activation and expansion of autologous NK cell using cytokines.

### Example 4

### In Vivo Migration of APCs to Draining Lymph Nodes

In vivo migration of APCs such as DCs, M1-macrophages, and B-cells, to draining lymph nodes can be measured in HBCx22 and HBCx34 patient-derived xenograft (PDX) mice models.

HBCx22 and HBCx34 PDX models have been established from untreated early-stage LBC as previously described (Cottu et al. Breast Cancer Res Treat 2012; 133:595-606). Both tumor models responded to endocrine therapy (ET). Luminal B status has been established on both patients' tumors and derived xenografts, and assessed on the basis of low PR/high Ki67 expression. To establish hormone-resistant models from these xenografts, tumor-bearing mice will be treated during 6 to 8 months with different ET, including tamoxifen. At tumor escape, resistant tumors will be re-engrafted in Swiss nude mice for three serial passages and treated with the therapy under which resistance will emerge. Resistant xenografts will be established when tumors have successfully undergone these three passages, and exhibited a resistance phenotype defined by a tumor growth pattern similar between the control group and the treated group.

In vivo migration of APCs to draining lymph nodes will be measured in these mice by injecting intraductally FITC-labeled CpG-ODN 2395 Vaccigrade™ or ODN 2395-FITC ((5′-tcgtcgttttcggcgc:gcgccg-3′) and OX-40 agonist antibody (InvivoMab clone OX-86 available from Bio-Cell) in to a breast milk duct of mice with a needle and syringe. After 24 hours, the lymph node under the armpit (axillary lymph node) will be dissected and measured for APCs by immunohistochemistry or flow cytometry as described in Example 4. The lymph nodes from ODN treated mice are expected to show higher levels of fully mature APCs (for example, one or more of the CCR7+, CD40+ CDOX40L+, CD80+, CD86+ and other mature APCs) compared to lymph nodes from control mice.

### Example 5

### Intraductal Administration of α-Type-1 Polarizing Bioactive Agents

Treatment Protocol. The primary objective of the study is to evaluate safety and tolerability of intraductal administration of an “αDC cocktail” of α-type-1 polarizing bioactive agents (TNFα/Il-1β/IFNγ/IFNα-2b/Poly I:C) in subjects with breast cancer in combination with ICD chemotherapy.

80 women with breast cancer will be recruited for the study and randomized to receive intraductally either placebo or a composition comprising the αDC cocktail (the “Drug”). The secondary object of the study is to evaluate (i) mobilization of immune cells, antigen presentation in the subject and (ii) preliminary efficacy by assessing, overall response rate and progression-free survival after treatment.

Subjects with breast cancer will be randomized to receive either treatment with placebo or the Drugs. All subject will receive chemotherapy with an ICD inducing cytotoxic agent oxaliplatin (85 mg/m² IV) on day 1 infused over 2 hours.

On day 1 following this pretreatment with cytotoxic agent, the subjects will receive either placebo or the Drug at doses in Table 3.

TABLE 3
αDC Cocktail Unit Dose

| Dose No. | TNFα:IL-β:IFNγ:IFNα-2b:Poly (I:C) |
|---|---|
| 1 | 1 μg/mL:1 ng/mL:100 U/mL:300 U/mL:2 μg/mL |
| 2 | 5 μg/mL:5 ng/mL:500 U/mL:1500 U/mL:10 μg/mL |
| 3 | 10 μg/mL:10 ng/mL:1000 U/mL:3000 U/mL:20 μg/mL |
| 4 | 20 μg/mL:20 ng/mL:2000 U/mL:6000 U/mL:40 μg/mL |

On day 8, subjects will again receive placebo or the Drug as described but without pretreatment with oxaliplatin. Placebo and the Drug will be administered intraductally as described above. This dosing regimen will be repeated every 2 weeks for at least 4 times. At the end of study period, the subjects will be monitored for additional 6 to 18 months.

On day 1 of the study dosing, prior to dosing with either placebo or the Drug, vital signs (systolic/diastolic blood pressure, pulse, temperature, respiratory rate) before dosing and at the following time points: 1, 2, 4, 8 and 12 hours following dose administration will be checked. A 12-lead ECG before dosing and at 4 hours following administration of the first dose will be performed. Blood samples for biomarker and pharmacokinetic (PK) analysis will be collected 10±1 minutes prior to dose administration to establish the baseline.

Starting day 8, vital signs (systolic/diastolic blood pressure, pulse, temperature, respiratory rate) will checked regularly on the day of each dosing (prior to dosing) during the study period. ECG, within 60 minutes prior to dose administration will also be performed, hematology, serum chemistry, coagulation and urinalysis and blood samples for PK analysis will be collected 10±1 minutes prior to administration of the study drug.

The safety profile and tolerability of each dose group will be determined by clinical assessment, subject reports and active follow-up throughout the study period. All adverse events, regardless of severity, will be recorded and evaluated.

Maturation of APCs such as DCs and mobilization of immune cells such as T-cells B-cells, and NK cells will be determined as described in Example 4. Migration of APCs to lymph nodes will be determined as described in Example 5. Subject's tumor size will be determined by mammography, CT-Scans and/or MM.

### Example 6

### In Vivo Migration of APCs to Draining Lymph Nodes

In resting DC, TLR3 and TLR9 are located in early endosomes and other intracellular compartments but migrates to LAMP1+ endosomes upon stimulation with ligands. TLR3 binds to TLR3 ligands such as a double-stranded RNA, for example, Poly I:C (e.g. rintatalomid) and TLR9 binds to ummethylated CpG DNA. In vivo migration of APCs such as DCs to draining lymph nodes can be measured in C57/B16 mice by delivering intraductally, for example, by injection, TLR3 ligand (Poly I:C HMW Fluorescein available from Invivogen) or TLR9 ligands CpG-ODN (5′-tcgtcgttttcggcgc:gcgccg-3′ (22 mer) available from Invivogen, and CCR7 ligands, CCL19 and CCL21 (available from R&D biosystems) in to a breast milk duct of mice with a needle and syringe. CCR7 ligands will be EGFP- or FITC-labeled to track the migration of APCs in vivo. After 24 hours, the lymph node under the armpit (axillary lymph node) will be dissected and measured for CCR7+ APCs by immunohistochemistry or flow cytometry.

### Example 7

### In Vivo Migration of APCs to Draining Lymph Nodes

In vivo migration of APCs such as DCs to draining lymph nodes can be measured in HBCx22 and HBCx34 patient derived xenograft (PDX) mice models as described above. One or more breast milk ducts of the PDX mice will be injected intraductally with TLR9 agonists FITC-labeled CpG-ODN 2395 Vaccigrade™ or ODN 2395-FITC ((5′-tcgtcgttttcggcgc:gcgccg-3′), and OX-40 agonist antibody (InvivoMab clone OX-86 available from Bio-Cell) with a needle and syringe. After 24 hours, the lymph node under the armpit (axillary lymph node) will be dissected and measured for CCR7+ APCs.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

All publications, patents and patent applications referred to herein are incorporated by reference in their entirety to the same extent as if each individual publication, patent or patent application was specifically and individually indicated to be incorporated by reference in its entirety.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of inducing immune response in a subject, comprising administering intraductally to a breast milk duct of the subject an effective amount of a composition comprising one or more bioactive agents, wherein the composition induces in situ maturation of antigen presenting cells.

2. The method of claim 1, wherein the one or more bioactive agents comprised in the composition is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U) and Polyinosinic-Polycytidylic acid stabilized with poly-L-lysine and carboxymethylcellulose (Poly-ICLC)); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7- and TLR8 agonists such as imidazoquinolines (Imiquimod and Resiquimod (R848)); TLR9 agonists such as (CpG-ODNs such as PF-3512676, and the like), DAMPs such as HMGB1, cytokines (such as TNFα, IFNγ, Type I IFNs such as IFNα or IFNβ, IL-1β, IL-2, IL-12), chemokines (such as IL-1β, CCL2, or CCR7 ligands such as CCL19, CCL21), and growth factors, mi-RNA such as miR-155, costimulatory molecule agonists (such as CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropyl-β-cyclodextrin, and a combination thereof.

3. The method of any one of the preceding claims, wherein the antigen presenting cell is a dendritic cell.

4. The method of any one of the preceding claims, wherein the composition induces migration of the antigen presenting cell to a lymph node in the subject.

5. The method of any one of the preceding claims, wherein the immune response comprises activation of effector T-cells, effector NK cells, effector B-cells, or a combination thereof.

6. The method of any one of the preceding claims, wherein the immune response comprises antitumor T-cell effector response, NK cell effector response, or B-cell anti-tumor effector response, or a combination thereof.

7. The method of any one of the preceding claims wherein the effector T-cells comprise cytotoxic CD8+ T-cells, CD4+ Th1 cells, memory T-cells, Tfh cells, or a combination thereof.

8. The method of any one of the preceding claims, wherein the immune response comprises reduction in immunosuppression.

9. The method of any one of the preceding claims, wherein the tumor size of the subject is reduced.

10. The method of any one of the preceding claims, wherein the composition further comprises an effective amount of a bioactive agent capable of inducing recruitment of inbound antigen presenting cells to the milk duct or breast tissue of the subject selected from the group consisting of cytokines and chemokines (such as IL-1β, MCP-1, RANTES, MIP-1α, MIP-1β, IL-8, C1q, CCL1 (CCR1 ligand), CCL2 (CCR2 ligand), CCLS (CCR5 ligand), CCL20 (CCR6 ligand), CXCL3 (CXCR3 ligand), CXCL4 (CXCR4 ligand) and CXCL1 (CXCR1 ligand)), DAMPs such as HMGB1, and TLR agonists (like TLR3 agonists (such as Poly (I:C), polyadenosine-polyuridylic acid (poly AU) Ampligen (polyI:polyC (12)U and Polyinosinic-Polycytidylic acid stabilized with poly-L-lysine and carboxymethylcellulose (Poly-ICLC), TLR4 agonists such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide, TLR7 agonists and TLR8 agonists such as imidazoquinolines (Imiquimod and Resiquimod (R848)), TLR9 agonists such as (CpG-ODNs such as PF-3512676, and the like).

11. The method of any one of the preceding claims, wherein the Type-1 polarizing agent or bioactive agent capable of inducing recruitment of inbound antigen presenting cells or both, is a TLR3 agonist, TLR4 agonist, TLR7 agonist, TLR8 agonist or TLR9 agonist.

12. The method of any one of the preceding claims, wherein the composition further comprises an effective amount of a repolarizing agent capable of repolarizing an M2-DC to Type-1 polarized DC (DC1) selected from the group consisting of fenretinide (4-hydroxy(phenyl)retinamide, 4-HPR); IL-12; IFNγ, miR127, miR155, and miR223, ferumoxytol, inhibitors of: CSF-1, CSF-1R, IL-10, IL-10R, TGFβ, Arginase 1 (Arg1), M2 macrophage scavenger receptors (such as A, B, MARCO), histone deacetylase (HDACi), DICER, IRF4/STAT4/STAT6 signaling pathway; IL-4, IL-13, IL-17, PPARγ, KLF4, KLF6, miRNA-146 family members such as (miRNA-146a), let7 family members (such as let-7c), miRNA-9, miRNA-21, miRNA-47, miRNA-187, CCR-CC12 axis signaling, CCL2/MCP-1, placental growth factor (PlGF) (HRG) and C/EBPβ (PI3Kγ deletion), AMPKα1 (metformin), p50-p50 NFκB, NADPH oxidase (NOX) (NOX 1 and NOX 2) such as GKT137831, Rbpj, Notch signaling pathway; activators/agonists of CD40 and CD40L, IRF1, IRF5, STAT1 (such as IFNγ, vadimezan (DMXAA)) and STAT3, nuclear factor kappa B activators, toll-like receptor (TLR) agonists of TLR3, TLR4, TLR7, TLR8, and TLR9 such as Imiquimod, synthetic unmethylated cytosine-guanine (CpG) oligodeoxinucleotides (CpG-ODNs), (poly I:C), C792, lefitolimod (MGN1703), SD-101 (Dynavax), SD-101, IMO-2125; p65-p50 NFκB, MyD88, miR127, miR155, and miR223, or a combination thereof.

13. The method of any one of the preceding claims, wherein the composition further comprises an effective amount of a blockading agent capable of reducing or preventing DC-to-macrophage shift, wherein the blockading agent is selected from a group consisting of CSF-1 inhibitors, CSF-1R inhibitors, MCP-1 inhibitors, IL-4 inhibitors (such as pascolizumab, pitakinra and dupilumab), IL-10 inhibitors, IL-13 inhibitors (such as anrukinzumab, lebrikizunab and tralokinumab), IL-4/IL-13 dual inhibitors such as duplimab, prostanoid inhibitors (such inhibitors of PGE3), STAT3 inhibitors (such as sorafenib, sunitinib, WP1066, and resveratrol), and STATE inhibitors (such as fenretinide (4-HPR), leflunomid, TMX264, and AS1217499), or a combination thereof.

14. The method of any one of the preceding claims, wherein the method further comprises administering to the subject an effective amount of an additional therapeutic agent selected from the group consisting of anti-hormonals (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubicin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubincin, idarubicin, mitoxantrone, oxaliplatin, paclitaxel, bleomycin, bortezomib, oncolytic viruses, patupilone, Tyrphostin AG 490, a Janus Activated Kinase 2/signal trasducer and activator of transcription-3 (JAK2/STAT3) inhibitor, DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inhibitors, anti-TGF-β inhibitors, checkpoint inhibitors (like PD-1 inhibitors such as anti-PD-1 antibodies (e.g., Nivolumab), PD-1L inhibitors such as anti-PD-1L (e.g., atezolizumab (MPDL3280), Avelumab (MSB0010718C), Durvalumab, MDX-1105), CTLA-4 inhibitors such as anti-CTLA4 antibodies (e.g., Ipilimumab), LAG-3 inhibitors such as anti-LAG-3 antibodies (e.g., IMP321, BMS-986016 and GSK2831781), OX-40 agonists such as MOXR0916, PF-04518600, MEDI0562, MEDI6469, and MEDI6383, TIM inhibitors, IDO inhibitors), CCR4 inhibitors, FoxP3 inhibitors, cell therapy such as Chimeric Antigen Receptor/T-cell (CAR-T) therapies, and other adoptive cell therapies, or a combination thereof.

15. The method of claim 14, wherein the additional therapeutic agent is comprised in any of the compositions of claims 1 to 13.

16. The method of claim 14 or claim 15, wherein the cytotoxic agent induces tumor cell death.

17. The method of any one of the preceding claims, wherein the subject is intraductally administered an effective amount of a composition comprising a TLR9 agonist and an OX-40 agonist.

18. The method of claim 17, wherein the TLR9 agonist is a CPG-ODN ranging from 0.01 μg/mL to 20 mg/mL, from 0.1 μg/mL to 15 mg/mL, from 1 μg/mL to 10 mg/mL, from 10 μg/mL to 5 mg/mL, or from 50 μg/mL to 1 mg/mL per unit dose, and the OX-40 agonist antibody ranges from 0.01 mg/mL to 50 mg/mL, 0.1 mg/mL to 40 mg/mL, 0.5 mg/mL to 30 mg/mL, or 1 mg/mL to 25 mg/mL per unit dose.

19. The method of any one of the preceding claims 1 to 18, wherein the subject is intraductally administered an effective amount of a composition comprising a TLR3 agonist and IFNα.

20. The method of claim 19, wherein the TLR3 agonist is Poly (I:C) ranging from 0.01 μg/mL to 50 μg/mL, from 0.1 μg/mL to 40 μg/mL, from 0.5 μg/mL to 25 μg/mL, or from 1 μg/mL to 20 μg/mL per unit dose, and the IFNα ranges from 1 μg/mL to 300 μg/mL, from 10 μg/mL to 250 μg/mL, from 25 μg/mL to 200 μg/mL, or from 50 μg/mL to 150 μg/mL per unit dose.

21. The method of any one of the preceding claims 1 to 16, wherein the subject is administered an effective amount of a composition comprising TNFα, IL-1β, IFNγ, IFNα-2b, and Poly (I:C).

22. The method of claim 21, wherein the TNFα ranges from 0.05 μg/mL to 150 μg/mL, from 0.1 μg/mL to 100 μg/mL, or from 0.5 μg/mL to 50 μg/mL per unit dose; IL-1β ranges from 0.01 μg/mL to 20 μg/mL, from 0.1 μg/mL to 15 μg/mL, from 0.5 μg/mL to 10 μg/mL, or from 1 μg/mL to 10 μg/mL per unit dose; IFNγ ranges from 1 μg/mL to 100 μg/mL, from 10 μg/mL to 80 μg/mL, from 25 μg/mL to 75 μg/mL, or from 50 μg/mL to 75 μg/mL per unit dose; IFNα from 1 μg/mL to 300 μg/mL, from 10 μg/mL to 250 μg/mL, from 25 μg/mL to 200 μg/mL, or from 50 μg/mL to 150 μg/mL per unit dose; and Poly (I:C) ranges from 0.01 μg/mL to 50 μg/mL, from 0.1 μg/mL to 40 μg/mL, from 0.5 μg/mL to 25 μg/mL, or from 1 μg/mL to 20 μg/mL per unit dose.

23. A method of inducing migration of antigen presenting cells in a subject comprising administering intraductally to a breast milk duct of the subject an effective amount of a composition comprising one or more bioactive agents, wherein at least one bioactive agent comprised in the composition is capable of inducing migration of the antigen presenting cells to a lymph node in the subject.

24. The method of claim 23, wherein the one or more bioactive agents is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U) and Polyinosinic-Polycytidylic acid stabilized with poly-L-lysine and carboxymethylcellulose (Poly-ICLC); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7 agonists and TLR8 agonists such as imidazoquinolines (Imiquimod and Resiquimod (R848); TLR9 agonists such as (CpG-ODNs such as PF-3512676, and the like), DAMPs such as HMGB1, cytokines (such as TNFα, IFNγ, Type I IFNs such as IFNα or IFNβ, IL-1β, IL-2, IL-12p70), DAMPs such as HMBG1, chemokines (such as IL-1β, MIP-3β, CCL2, CCL19, CCL21 or any CCR7 ligands), and growth factors, mi-RNA such as miR-155, costimulatory molecule agonists (such as CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropyl-β-cyclodextrin, and a combination thereof.

25. The method of claim 23 or 24, wherein the at least one bioactive agent capable of inducing migration of the antigen presenting cells to a lymph node in the subject is IL-1β, MIP-3β, CCL2, CCR7 ligand such as CCL19 and CCL21, LMP1, LMP1-CD40, LMP1-OX40 agonist, CD40L, MMP9, DAMPs such as HMBG1, or a combination thereof.

26. The method of any one of claims 23 to 25, wherein the antigen presenting cell is a dendritic cell.

27. The method of any one of claims 23 to 26, wherein the antigen presenting cells migrating to the lymph node activates cytotoxic CD8+ T-cells, CD4+ Th1 cells, memory T-cells, memory B-cells, Thf cells, NK cells, B-cells or any combination thereof.

28. The method of any one of claims 23 to 27, wherein the method induces an anti-tumor immune response in the subject.

29. The method of any one of claims 23 to 28, wherein anti-tumor immune response comprises breast tumor infiltration by activated cytotoxic CD8+ T-cells, CD4+Th1 cells, NK cells, B-cells or a combination thereof.

30. The method of any one of claims 23 to 29, wherein the size of subject's breast tumor is reduced.

31. A method for inducing or augmenting immunological cell death in breast tumor cells of a subject, comprising administering to the subject an effective amount of a cytotoxic agent, and administering intraductally an effective amount of a composition comprising one or more bioactive agents.

32. The method of claim 31, wherein the cytotoxic agent is selected from the group consisting of temozolomide, cyclophosphamide (including low dose or metronomic cyclophosphamide), mafosfamide, doxorubicin, epirubicin, idarubicin, mitoxantrone, oxaliplatin, paclitaxel, bleomycin, bortezomib, oncolytic viruses, patupilone, tyrphostin AG 490 (JAK2/STAT3 inhibitor), or a combination thereof.

33. The method of claim 32, wherein the one or more bioactive agents is a Type 1-polarizing agent selected from the group consisting of TLR agonists (e.g., TLR3 agonists (such as Poly (I:C), polyadenosine-polyuridylic acid (poly AU) Ampligen (polyI:polyC (12)U) and Polyinosinic-Polycytidylic acid stabilized with poly-L-lysine and carboxymethylcellulose (Poly-ICLC); TLR4 agonists (such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide); TLR7 agonists and TLR8 agonists such as imidazoquinolines (Imiquimod and Resiquimod (R848)); TLR9 agonists such as (CpG-ODNs such as PF-3512676, and the like), DAMPs such as HMGB1, cytokines (such as TNFα, IFNγ, Type I IFNs such as IFNα or IFNβ, IL-1β, IL-2, IL-12), chemokines (such as IL-1β, CCL2, CCL19, CCL21 or any CCR7 ligands), and growth factors, mi-RNA such as miR-155, costimulatory molecule agonists (such as CD-40 agonists (e.g. anti-CD40 antibodies such as R07009789, APX005M, CP-870,893, ABBV-927), OX-40 agonists (e.g. anti-OX-40 antibodies MOXR0916, PF-04518600, MEDI0562, MEDI6469, and MEDI6383), cyclodextrins such as 2-hydroxypropyl-β-cyclodextrin, and a combination thereof.

34. The method of any one of claims 31 to 33, wherein the method further comprises intraductal administration of an effective amount of a bioactive agent capable of inducing recruitment of inbound antigen presenting cells to the milk duct or breast tissue of the subject selected from the group consisting of cytokines and chemokines such as IL-1β, MCP-1, RANTES, MIP-1α, MIP-1β, IL-8, C1Q, CCL1 (CCR1 ligand), CCL2 (CCR2 ligand), CCL5 (CCR5 ligand), CCL20 (CCR6 ligand), CXCL3 (CXCR3 ligand), CXCL4 (CXCR4 ligand) and CXCL1 (CXCR1 ligand), DAMPs such as HMGB1, and TLR agonists (like TLR3 agonists (such as Poly (I:C), polyadenosine-polyuridylic acid (poly AU) Ampligen (polyI:polyC (12)U; and Polyinosinic-Polycytidylic acid stabilized with poly-L-lysine and carboxymethylcellulose (Poly-ICLC), TLR4 agonists such as glucanopynosyl lipoid A (G100), GSK1795091, Monophosphoryl lipid A (MPL) and MPL-based agonists such as aminoalkyl glucosaminide phosphates (AGPs), lipopolysaccharides (LPS) and opioids such as methadone, morphine-3-glucuronide, TLR7 agonists and TLR8 agonists such as imidazoquinolines (Imiquimod and Resiquimod (R848), TLR9 agonists such as (CpG-ODNs such as PF-3512676, (poly I:C), C792, lefitolimod (MGN1703), SD-101, IMO-2125, and the like), or a combination thereof.

35. The method of any one of claims 31 to 34, wherein the cytotoxic agent comprises oxaliplatin, and wherein the composition comprising one or more bioactive agents comprises (i) a TLR3 agonist poly (I:C) and IFNα; (ii) a TLR9 agonist (CpG-ODNs) and OX-40 agonist antibody; or (iii) TNFα, IL-1β, IFNγ, IFNα-2b, and Poly (I:C).

36. The method of any one of claims 31 to 35, wherein cytotoxic agent is administered to the subject by intravenously or intraductally.

37. The method of any one of claims 31 to 36, wherein the tumor size of the subject is reduced.

38. The method of any of the preceding claims, wherein the composition is intraductally administered in a single dose or multiple doses.

39. The method of any one of the preceding claims, wherein the composition is administered daily, several times a day (twice, thrice, four times and the like), alternate days, every 2 days, 3 days, 5 days, 7 days, 14 days, 15 days, every 3 weeks, 28 days, monthly, quarterly, 6 monthly, and annually.

40. The composition of any one of the preceding claims, further comprising an imaging agent, a dye or a contrasting agent selected from the groups consisting of gadolinium chelates, superparamagnetic iron oxide nanoparticles (SPION), ¹⁹F perfluorocarbon nanoparticles, and other magnetic reporter genes, such as metalloprotein-based MM probes.

41. The composition of any one of the preceding claims, further comprising a pharmaceutically acceptable carrier.

42. The composition of any one of the preceding claims, wherein the composition is formulated as a liposome, a nanoparticle, a microparticle, a microsphere, a nanocapsule, a nanosphere, a lipid particle, a vesicle, or a micelle.

43. The composition of any one of the preceding claims, wherein the one or more bioactive agent is comprised in a liposome, a microparticle, a microsphere, a nanocapsule, a nanoparticle, a nanosphere, a lipid particle, a vesicle, or a micelle.

44. The composition of any one of the preceding claims, wherein the one or more bioactive agent is comprised on a liposome, a microparticle, a microsphere, a nanocapsule, a nanoparticle, a nanosphere, a lipid particle, a vesicle, or a micelle.

45. The composition of any one of the preceding claims, wherein the nanoparticle is a lipid nanoparticle.

46. The composition of any one of the preceding claims, wherein the nanoparticle is further coated with a cell targeting agent.

47. The composition of claim 46, wherein the cell targeting agent is selected from the group consisting of DEC-205, Clec9A (DNGR-1), DC-SIGN, C1q, BDCA1, BDCA2, BDCA3 and BDCA4.

48. An article of manufacture, comprising a composition according to any of claims 1 to 47, one or more containers, packaging material, a label or package insert, and optionally, a device.

49. The article of claim 48, wherein the device is a needle and syringe, a cannula, a catheter, a microcatheter, an osmotic pump, or an encapsulation device.

50. The article of claim 48 or claim 49, further comprising an additional therapeutic agent selected from the group consisting of checkpoint inhibitors, anti-hormonals (e.g., anti-estrogen or anti-estrogen receptor, such as tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole), steroids, anthracyclines, thyroid hormone replacement drugs, cytotoxic agents such as alkylating agents (such as temozolomide and cyclophosphamide), anthracyclines (such as doxorubicin, pegylated liposomal doxorubicin, epirubicin, idarubicin, and the like), anthracenediones such as mitoxantrone, platinum drugs (such as cisplatin, carboplatin, oxaliplatin, ormaplatin, enloplatin, and the like), taxanes (such as paclitaxel), antimitotic drugs, bleomycin, bortezomib, patupilone, calreticulin, broad spectrum cell death agents such as glossypol, tea phenols such as Epigallocatechin-3-Gallate, 7-Bromoindirubin-3′-oxime (7BIO)-, oncogenic RAS, macrolides, Berberine (an isoquinoline alkaloid derived from plants), UMI-77, triptolide and selinexor, broad spectrum inhibitor of extracellular nucleotidases, such as ARL67156, temozolomide cyclophosphamide, mafosfamide, doxorubicin, epirubicin, idarubicin, mitoxantrone, oxaliplatin, paclitaxel, bleomycin, bortezomib, oncolytic viruses, patupilone, Tyrphostin AG 490 (JAK2/STAT3 inhibitor), DNA hypomethylating agents (such as azacitidine or decitabine), thymidylate-targeted drugs (such as docetaxel, gemcitabine), trastuzumab, ado-trastuzumab emtansine, pertuzumab, abemaciclib, palbociclib, anti-IL-10 inhibitors, anti-TGF-β inhibitors, checkpoint inhibitors (such as anti-PD-1 antibodies, anti-PD-1L antibodies, anti-PD-L2 antibodies, anti-CTLA-4 antibodies, anti-LAG-3 antibodies, anti-TIM-3 antibodies, and the like), anti-CCR4 inhibitors, anti-FoxP3 inhibitors, cell therapy such as Chimeric Antigen Receptor/T-cell (CAR-T) therapies, and other adoptive cell therapies, or a combination thereof.
